(19) United States
(12) Patent Application Publication
(10) Pub. No.: US 2008/0182874 A1
(43) Pub. Date: Jul. 31, 2008

(54) Novel Compounds

(75) Inventors: Richard Evans, Leicestershire (GB); Rhonan Ford, Leicestershire (GB); Toby Thompson, Leicestershire (GB); Paul Willis, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/720,359

(22) PCT Filed: Nov. 28, 2005
(86) PCT No.: PCT/SE05/01782

§ 371 Date: May 29, 2007

(30) Foreign Application Priority Data
Nov. 30, 2004 (SE) 0402925-2

(51) Int. Cl. A61K 31/4709; C07D 215/12; A61K 31/47; A61P 11/06; A61P 19/02; A61P 11/00; C07D 401/04

(52) U.S. Cl. 514/314; 546/165; 514/311

(57) ABSTRACT

The invention provides compounds of formula (I), or a pharmaceutically acceptable salt or an in vivo hydrolysable ester thereof, in which A, n, p, q, R¹, R², R³ and R⁴ are as defined in the specification; a process for their preparation; pharmaceutical compositions containing them; and their use in therapy.

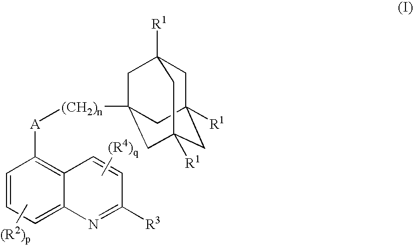

The present invention relates to adamantane derivatives, processes for their preparation, pharmaceutical compositions containing them, a process for preparing the pharmaceutical compositions, and their use in therapy.

The P2X₇ receptor (previously known as P2Z receptor), which is a ligand-gated ion channel, is present on a variety of cell types, largely those known to be involved in the inflammatory/immune process, specifically, macrophages, mast cells and lymphocytes (T and B). Activation of the P2X₇ receptor by extracellular nucleotides, in particular adenosine triphosphate, leads to the release of interleukin-1β (IL-1β) and giant cell formation (macrophages/microglial cells), degranulation (mast cells) and proliferation (T cells) and apoptosis and L-selectin shedding (lymphocytes). P2X₇ receptors are also located on antigen-presenting cells (APC), keratinocytes, salivary acinar cells (parotid cells), hepatocytes and mesangial cells.

It would be desirable to make compounds effective as P2X₇ receptor antagonists for use in the treatment of inflammatory, immune or cardiovascular diseases, in the aetiologies of which the P2X₇ receptor may play a role.

The present invention provides a new class of adamantyl-containing P2X₇ antagonist which comprise a substituted quinoline moiety. These novel compounds display attractive properties for use as P2X₇ receptor antagonists in the treatment of inflammatory, immune or cardiovascular diseases. Whilst adamantyl-containing P2X₇ antagonists have been described previously, for example in WO 99/29661, WO 99/29660, WO 00/61569 and WO 03/080579, prior to the present invention there had been no suggestion that a compound comprising the substituted quinoline moiety of the present invention would make an effective P2X₇ antagonist. Whilst WO 03/080579 does describe some quinoline-containing compounds, it does not teach the use of a quinoline moiety bearing the substituents specified in the present invention.

In accordance with the present invention, there is therefore provided a compound of formula

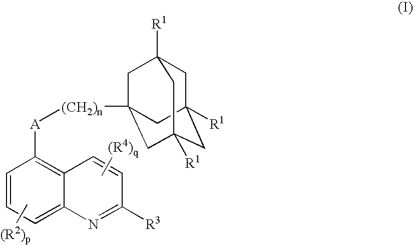

or a pharmaceutically acceptable salt or an in vivo hydrolysable ester thereof, wherein n represents 1, 2 or 3;

each R¹ independently represents hydrogen, hydroxy or a halogen;

A is C(O)NH or NHC(O);

p is 0, 1 or 2;

each R² independently represents halogen or C₁₋₆ alkyl optionally substituted by at least one substituent independently selected from hydroxyl, halogen and C₁₋₆ alkoxy;

q is 0, 1 or 2;

each R⁴ independently represents halogen or C₁₋₆ alkyl optionally substituted by at least one substituent independently selected from hydroxyl, halogen and C₁₋₆alkoxy;

R³ represents a group Y¹R⁶ or Z¹R¹⁰;

R⁶ represents a group R⁸ or a 4- to 9-membered carbocyclic or heterocyclic ring, which carbocyclic or heterocyclic ring is substituted by at least one substituent independently selected from Y²R⁹ and Z²R¹¹, and which 4- to 9-membered carbocyclic or heterocyclic ring may further be optionally substituted by at least one substituent independently selected from halogen, hydroxyl, C₁₋₆alkoxy, C₁₋₆ alkyl, phenyl and a 5- to 6-membered heteroaromatic ring, which C₁₋₆ alkyl, phenyl or 5- to 6-membered heteroaromatic ring may be optionally substituted by at least one substituent selected from halogen, hydroxyl and C₁₋₆alkoxy;

R⁸ and R⁹ each independently represent tetrazolyl, 1,2,3-triazolyl, 1,2,4-triazolyl or a 5- to 6-membered heterocyclic ring comprising from 1 to 4 heteroatoms independently selected from nitrogen, oxygen and sulphur, which heterocyclic ring is substituted by at least one substituent selected from hydroxyl, ═O and ═S, and which heterocyclic ring may further be optionally substituted by at least one substituent selected from halogen, nitro, amino, cyano, C₁₋₆ alkylsulphonyl, C₁₋₆alkoxycarbonyl and a C₁₋₆ alkyl group which C₁₋₆ alkyl group can be optionally substituted by at least one substituent selected from halogen, hydroxyl and amino;

R¹⁰ and R¹¹ each independently represent carboxyl, C₁₋₆ alkylsulphonylaminocarbonyl C(O)NHOH or NHR¹²; R¹² represents CN, C₁₋₆ alkylsulphonyl, C₁₋₆ alkylcarbonyl, C₁₋₆ alkoxycarbonyl, C₁₋₆ alkylaminosulphonyl or (di)-C₁₋₆ alkylaminosulphonyl;

Y¹ and Y² each independently represent a bond, O, S(O)₀₋₂, NR⁷C(O), C(O)NR⁷, SO₂NR⁷, NR⁷SO₂, >NR⁷, O(CH₂)₁₋₆, S(O)₀₋₂(CH₂)₁₋₆, NR⁷(CH₂)₁₋₆, (CH₂)₁₋₃O(CH₂)₁₋₃, (CH₂)₁₋₃S(O)₀₋₂(CH₂)₁₋₃, (CH₂)₁₋₃NR⁷(CH₂)₁₋₃, (CH₂)₁₋₃NR⁷C(O)(CH₂)₀₋₃, (CH₂)₁₋₃C(O)NR⁷(CH₂)₀₋₃, S(O)₀₋₂(CH₂)₁₋₆NR⁷ or a C₁₋₆ alkylene which C₁₋₆ alkylene can be optionally substituted by at least one substituent independently selected from hydroxyl, halogen and C₁₋₆alkoxy;

Z¹ and Z² each independently represent a bond, O(CH₂)₁₋₆, S(O)₀₋₂(CH₂)₁₋₆, NR⁷(CH₂)₁₋₆, (CH₂)₁₋₃O(CH₂)₁₋₃, (CH₂)₁₋₃S(O)₀₋₂(CH₂)₁₋₃, (CH₂)₁₋₃NR⁷(CH₂)₁₋₃, (CH₂)₁₋₃NR⁷C(O)(CH₂)₁₋₃, (CH₂)₁₋₃C(O)NR⁷(CH₂)₁₋₃ or a C₁₋₆ alkylene which C₁₋₆ alkylene can be optionally substituted by at least one substituent independently selected from hydroxyl, halogen and C₁₋₆alkoxy; and

each R⁷ independently represents hydrogen or a C₁₋₆ allyl group which can be optionally substituted by at least one substituent independently selected from hydroxyl, halogen and C₁₋₆alkoxy;

with the provisos that:
(a) when R³ represents Y¹R⁶ and Y¹ represents NR⁷(CH₂)₁₋₆, S(CH₂)₁₋₆, O(CH₂)₁₋₆ or an optionally substituted C₁₋₆ alkylene, then R⁶ does not represent oxopyrrolidinyl;
(b) when R³ represents Z¹R¹⁰ and Z¹ represents (CH₂)₁₋₃NR⁷(CH₂)₁₋₃, then R¹⁰ does not represent carboxyl;
(c) when R³ represents Y¹R⁶ and Y¹ represents (CH₂)₁₋₃NR⁷(CH₂)₁₋₃ and R⁶ represents a group R⁸, then R⁸ does not represent a 5- to 6-membered heterocyclic ring substituted by hydroxyl or ═O;
(d) when R³ represents Y¹R⁶ and Y¹ represents (CH₂)₁₋₃NR⁷(CH₂)₁₋₃ and R⁶ represents a 4- to 9-membered carbocyclic or heterocyclic ring substituted by Z²R¹¹ and Z² represents a bond, then R¹¹ does not represent carboxyl;
(e) when R³ represents Y¹R⁶ and Y¹ represents NR⁷(CH₂)₁₋₆, S(CH₂)₁₋₆, O(CH₂)₁₋₆ or an optionally substituted C₁₋₆ alkylene and R⁶ represents phenyl substituted by Z²R¹¹ and Z² represents a bond, then R¹¹ does not represent C₁₋₆ alkylsulphonylamino;
(f) when R³ represents Z¹R¹⁰ and Z¹ represents O(CH₂)₁₋₆, S(CH₂)₁₋₆, NR⁷(CH₂)₁₋₆ or an optionally substituted C₁₋₆ alkylene and R¹⁰ represents NHR¹², then R¹² does not represent C₁₋₆ alkylcarbonyl; and
(g) the compound is not selected from tert-butyl 1-{5-[(1-adamantylacetyl)amino]-6-methylquinolin-2-yl}piperidin-4-ylcarbamate and tert-butyl (3S)-1-{5-[(1-adamantylacetyl)amino]-6-methylquinolin-2-yl}pyrrolidin-3-ylcarbamate.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

It will be understood that certain compounds of the present invention may exist in solvated, for example hydrated, as well as unsolvated forms. It is to be understood that the present invention encompasses all such solvated forms.

In the context of the present specification, a “Carbocyclic” ring is an unsaturated, saturated or partially saturated mono- or bicyclic ring, containing only carbon ring atoms, and may have aliphatic or aromatic properties. A “Heterocyclic” ring is an unsaturated, saturated or partially saturated mono- or bicyclic ring, at least one atom of which is a heteroatom selected from oxygen, sulphur or nitrogen, and may have aliphatic or aromatic properties. The expression “Heteroaromatic” denotes aromatic rings, at least one atom of which is a heteroatom selected from oxygen, sulphur or nitrogen. Unless otherwise indicated an alkyl group may be linear or branched.

In an embodiment of the invention, n represents 1.

In an embodiment of the invention, each R¹ independently represents a hydrogen atom.

In an embodiment of the invention, A represents C(O)NH. In an alternative embodiment of the invention, A represents NH(CO).

In an embodiment of the invention, p is 0 or 1.

Each R² independently represents halogen (e.g. chlorine, fluorine, bromine or iodine) or C₁₋₆, preferably C₁₋₄, alkyl (e.g. methyl, ethyl, n-propyl, isopropyl, n-butyl, isobutyl, tert-butyl, n-pentyl or n-hexyl) optionally substituted by at least one substituent (e.g. zero, one, two or three) independently selected from hydroxyl, halogen (e.g. chlorine, fluorine, bromine or iodine) and C₁₋₆, preferably C₁₋₄, alkoxy (e.g. methoxy, ethoxy, n-propoxy or n-butoxy).

In an embodiment of the invention, each R² independently represents halogen or C₁₋₄ alkyl.

In an embodiment of the invention, q is 0 or 1.

Each R⁴ independently represents halogen (e.g. chlorine, fluorine, bromine or iodine) or C₁₋₆, preferably C₁₋₄, alkyl (e.g. methyl, ethyl, n-propyl, isopropyl, n-butyl, isobutyl, tert-butyl, n-pentyl or n-hexyl) optionally substituted by at least one substituent (e.g. zero, one, two or three) independently selected from hydroxyl, halogen (e.g. chlorine, fluorine, bromine or iodine) and C₁₋₆, preferably C₁₋₄, alkoxy (e.g. methoxy, ethoxy, n-propoxy or n-butoxy).

In an embodiment of the invention, each R⁴ independently represents halogen or C₁₋₄ alkyl.

R⁶ represents a group R⁸ or a 4- to 9-membered carbocyclic or heterocyclic ring, which carbocyclic or heterocyclic ring is substituted by at least one substituent (e.g. one or two) independently selected from Y²R⁹ and Z²R¹¹, and which 4- to 9-membered carbocyclic or heterocyclic ring may further be optionally substituted by at least one substituent (e.g. zero, one or two) independently selected from halogen (e.g. chlorine, fluorine, bromine or iodine), hydroxyl, C₁₋₆, preferably C₁₋₄, alkoxy (e.g. methoxy, ethoxy, n-propoxy or n-butoxy), C₁₋₆, preferably C₁₋₄, alkyl (e.g. methyl, ethyl, n-propyl, isopropyl, n-butyl, isobutyl, tert-butyl, n-pentyl or n-hexyl), phenyl and a 5- to 6-membered heteroaromatic ring, which C₁₋₆ alkyl, phenyl or 5- to 6-membered heteroaromatic ring may be optionally substituted by at least one substituent (e.g. zero, one, two or three) selected from halogen (e.g. chlorine, fluorine, bromine or iodine), hydroxyl and C₁₋₆ preferably C₁₋₄, alkoxy (e.g. methoxy, ethoxy, n-propoxy or n-butoxy).

When R⁶ represents a 4- to 9-membered carbocyclic ring, examples of carbocyclic rings include cyclobutyl, cyclopentyl, cyclohexyl and phenyl.

In an embodiment of the invention, R⁶ represents phenyl. In a further aspect of this embodiment the phenyl group is substituted with a group Z²R¹¹, wherein Z² represents a bond or a C₁₋₆ alkylene group and R¹¹ represents carboxyl.

When R⁶ represents a 4- to 9-membered heterocyclic ring, examples of heterocyclic rings are rings containing from 1 to 3, or 1 to 2, heteroatoms selected from nitrogen, oxygen and sulphur.

In an embodiment of the invention, R⁶ represents an aromatic 5- to 8-membered heterocyclic ring containing from 1 to 3, or 1 to 2, heteroatoms selected from nitrogen, oxygen and sulphur. Examples of aromatic heterocyclic rings according to this embodiment include pyridinyl, pyridazinyl, pyrizinyl, pyrimidyl, pyrazolyl.

In an embodiment of the invention, R⁶ represents an aliphatic 5- to 8-membered heterocyclic ring containing one nitrogen atom and optionally one further heteroatom selected from nitrogen and oxygen. Examples of heterocyclic rings according to this embodiment include pyrrolidinyl, piperidinyl, piperazinyl, homopiperazinyl, homopiperidinyl and azabicyclo[3.1.0]hexanyl.

R⁸ and R⁹ each independently represent tetrazolyl, 1,2,3-triazolyl, 1,2,4-triazolyl or a 5- to 6-membered heterocyclic ring comprising from 1 to 4 heteroatoms independently selected from nitrogen, oxygen and sulphur, which heterocyclic ring is substituted by at least one substituent (e.g. zero, one, two or three) selected from hydroxyl, ═O and ═S, and which heterocyclic ring may further be optionally substituted by at least one substituent (e.g. zero, one or two) selected from halogen (e.g. chlorine, fluorine, bromine or iodine), nitro, amino, cyano, C₁₋₆, preferably C₁₋₄, alkylsulphonyl (e.g. MeSO₂— or EtSO₂—), C₁₋₆, preferably C₁₋₄, alkoxycarbonyl (e.g. methoxy-, ethoxy-, n-propoxy-, n-butoxy-, n-pentoxy- or n-hexoxycarbonyl), and a C₁₋₆, preferably C₁₋₄, alkyl (e.g. methyl, ethyl, n-propyl, isopropyl, n-butyl, isobutyl, tert-butyl, n-pentyl or n-hexyl) group which C₁₋₆ alkyl group can be optionally substituted by at least one substituent (e.g. zero, one, two or three) selected from halogen (e.g. chlorine, fluorine, bromine or iodine), hydroxyl and amino.

When R⁸ and R⁹ each independently represent a 5- to 6-membered heterocyclic ring, nitrogen heteroatoms in the heterocyclic ring may carry hydroxyl substituents and sulphur atoms in the ring may be in the form of S, SO (i.e. carrying one ═O substituent) or SO₂ (i.e. carrying two ═O substituents).

Where R⁹ or R¹⁰ represents a 5- to 6-membered heterocyclic ring comprising from 1-4 heteroatoms independently selected from nitrogen, oxygen and sulphur, which heterocyclic ring is substituted by at least one substituent selected from hydroxyl, ═O and ═S, examples include:

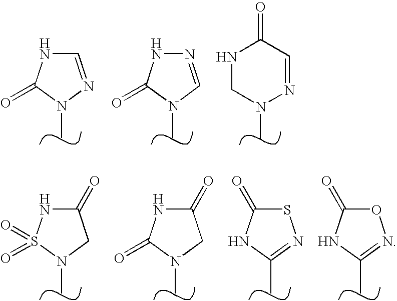

In an embodiment of the invention, R⁸ and R⁹ each independently tetrazolyl, 1,2,3-triazolyl, 1,2,4-triazolyl,

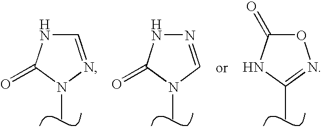

R¹⁰ and R¹¹ each independently represent carboxyl, C₁₋₆ alkylsulphonylaminocarbonyl (e.g. MeSO₂NHCO—, or EtSO₂NHCO—), C(O)NHOH, NHR¹²; R¹² represents CN, C₁₋₆, preferably C₁₋₄, alkylsulphonyl (e.g. MeSO₂— or EtSO₂—), C₁₋₆, preferably C₁₋₄, alkylcarbonyl (e.g. methyl-, ethyl-, n-propyl-, n-butyl-, n-pentyl- or n-hexylcarbonyl), C₁₋₆, preferably C₁₋₄, alkoxycarbonyl (e.g. methoxy-, ethoxy-, n-propoxy-, n-butoxy-, n-pentoxy- or n-hexoxycarbonyl), C₁₋₆, preferably C₁₋₄, alkylaminosulphonyl (e.g. MeNHSO₂ or EtNHSO₂—), or (di)-C₁₋₆, preferably C₁₋₄, alkylaminosulphonyl (e.g. Me₂NSO₂— or Et₂NSO₂— or EtMeNSO₂—).

In an embodiment of the invention, R¹⁰ and R¹¹ each independently represent carboxyl, C₁₋₆ alkylsulphonylaminocarbonyl or C(O)NHOH.

In another embodiment of the invention, R¹⁰ and R¹¹ each independently represent carboxyl.

In an embodiment of the invention, Y¹ and Y² each independently represent a bond, O, S(O)₀₋₂, NR⁷C(O), C(O)NR⁷, SO₂NR⁷, NR⁷SO₂, >NR⁷, O(CH₂)₁₋₆, S(O)₀₋₂(CH₂)₁₋₆, NR⁷(CH₂)₁₋₆, (CH₂)₁₋₃O(CH₂)₁₋₃, (CH₂)₁₋₃S(O)₀₋₂(CH₂)₁₋₃, (CH₂)₁₋₃NR⁷C(O)(CH₂)₀₋₃, (CH₂)₁₋₃C(O)NR⁷(CH₂)₀₋₃, S(O)₀₋₂(CH₂)₁₋₆NR⁷ or a C₁₋₆, preferably C₁₋₄, alkylene group which C₁₋₆ alkylene can be optionally substituted by at least one substituent (e.g. zero, one, two or three) independently selected from hydroxyl, halogen (e.g. chlorine, fluorine, bromine or iodine), and C₁₋₆, preferably C₁₋₄, alkoxy (e.g. methoxy, ethoxy, n-propoxy or n-butoxy).

In another embodiment of the invention, Y¹ and Y² each independently represent a bond, O, S(O)₀₋₂, >NR⁷, O(CH₂)₁₋₆, NR⁷(CH₂)₁₋₆ or a C₁₋₆ alkylene.

In an embodiment of the invention, Z¹ and Z² each independently represent a bond, O(CH₂)₁₋₆, S(O)₀₋₂(CH₂)₁₋₆, NR⁷(CH₂)₁₋₆, (CH₂)₁₋₃O(CH₂)₁₋₃, (CH₂)₁₋₃S(O)₀₋₂(CH₂)₁₋₃, (CH₂)₁₋₃NR⁷(CH₂)₁₋₃, (CH₂)₁₋₃NR⁷C(O)(CH₂)₁₋₃, (CH₂)₁₋₃C(O)NR⁷(CH₂)₁₋₃ or a C₁₋₆, preferably C₁₋₄, alkylene group which C₁₋₆ alkylene can be optionally substituted by at least one substituent (e.g. zero, one, two or three) independently selected from hydroxyl, halogen (e.g. chlorine, fluorine, bromine or iodine), and C₁₋₆, preferably C₁₋₄, alkoxy (e.g. methoxy, ethoxy, n-propoxy or n-butoxy).

In another embodiment of the invention Z¹ and Z² each independently represent a bond, O(CH₂)₁₋₆, NR⁷(CH₂)₁₋₆ or a C₁₋₆ alkylene.

Each R⁷ independently represents a hydrogen atom or a C₁₋₆, preferably C₁₋₄, alkyl (e.g. methyl, ethyl, n-propyl, isopropyl, n-butyl, isobutyl, tert-butyl, n-pentyl or n-hexyl) group which can be optionally substituted by at least one substituent (e.g. zero, one, two or three) independently selected from hydroxyl, halogen (e.g. fluorine, chlorine, bromine or iodine) and C₁₋₆, preferably C₁₋₄, alkoxy (e.g. methoxy, ethoxy, n-propoxy, n-butoxy, n-pentoxy or n-hexoxy).

An embodiment of the invention provides a compound of formula

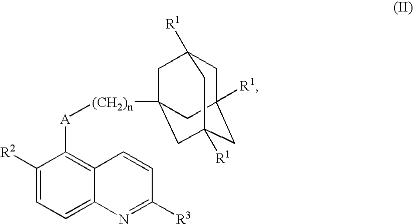

or a pharmaceutically acceptable salt or an in vivo hydrolysable ester thereof, wherein A, n, R¹, R² and R³ are as defined in formula (I).

In an embodiment of the invention, R³ represents a group Y¹R⁶. In a further aspect of this embodiment, Y¹ represents a bond or a C₁₋₆ alkylene and R⁶ represents a group R⁸ or a 4- to 9-membered heterocyclic ring substituted by at least one substituent selected from Y²R⁹ and Z²R¹¹, which 4- to 9-membered heterocyclic ring can further be optionally substituted by a substituent selected from halogen, hydroxyl, C₁₋₄ allyl and phenyl. In a still further aspect of this embodiment, Z² represents a bond, NR⁷(CH₂)₁₋₆ or a C₁₋₆ alkylene group and R¹¹ represents carboxyl, C₁₋₆ alkylsulphonylaminocarbonyl or C(O)NHOH. In another aspect of this embodiment, Y² represents a bond, S(O)₀₋₂, NR⁷(CH₂)₁₋₆ or a C₁₋₆ alkylene group, and R⁹ represents tetrazolyl, 1,2,3-triazolyl, 1,2,4-triazolyl,

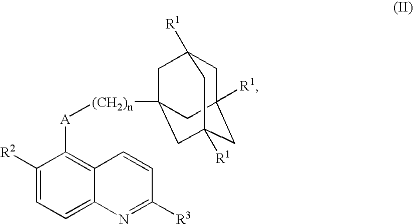

In another embodiment of the invention, R³ represents a group Y¹R⁶ wherein Y¹ represents a bond.

In a further embodiment of the invention, there is provided a subset of compounds of formula (I) and pharmaceutically acceptable salts or in vivo hydrolysable esters thereof, wherein

n represents 1;

each R¹ independently represents hydrogen;

A is C(O)NH or NHC(O);

p is 0 or 1;

each R² independently represents halogen or C₁₋₄ alkyl;

q is 0 or 1;

each R⁴ independently represents halogen or C₁₋₄ alkyl;

R³ represents a group Y¹R⁶;

R⁶ represents a group R⁸ or a 4- to 9-membered carbocyclic or heterocyclic ring, which carbocyclic or heterocyclic ring is substituted by at least one substituent independently selected from Y²R⁹ and Z²R¹¹, and which 4- to 9-membered carbocyclic or heterocyclic ring may further be optionally substituted by at least one substituent independently selected from halogen, hydroxyl, C₁₋₄ alkyl and phenyl;

R⁸ and R⁹ each independently represent tetrazolyl, 1,2,3-triazolyl, 1,2,4-triazolyl, or a 5- to 6-membered heterocyclic ring comprising from 1 to 4 heteroatoms independently selected from nitrogen, oxygen and sulphur, which heterocyclic ring is substituted by at least one substituent selected from hydroxyl, ═O and ═S;

R¹¹ represents carboxyl, C₁₋₆ alkylsulphonylaminocarbonyl or C(O)NHOH;

Y¹ and Y² each independently represent a bond, O, S(O)₀₋₂, >NR⁷, O(CH₂)₁₋₆, NR⁷(CH₂)₁₋₆ or a C₁₋₆ alkylene;

Z² represents a bond, O(CH₂)₁₋₆, NR⁷(CH₂)₁₋₆ or a C₁₋₆ alkylene; and each R⁷ independently represents hydrogen or a C₁₋₄ alkyl group;

subject to proviso (a) of formula (I).

In a still further embodiment of the invention, there is provided a subset of compounds of formula (I) and pharmaceutically acceptable salts or in vivo hydrolysable esters thereof, wherein

n represents 1;

each R¹ independently represents hydrogen;

A is C(O)NH or NHC(O);

p is 0 or 1;

each R² independently represents halogen or C₁₋₄ alkyl;

q is 0 or 1;

each R⁴ independently represents halogen or C₁₋₄ alkyl;

R³ represents a group Y¹R⁶;

R⁶ represents a cyclic group selected from phenyl, pyridinyl or a 5- to 8-membered heterocyclic ring containing one nitrogen atom and optionally one further heteroatom selected from nitrogen and oxygen (e.g. pyrrolidinyl, piperidinyl, piperazinyl, homopiperazinyl, homopiperidinyl and azabicyclo[3.1.0]hexanyl), which cyclic group R⁶ is substituted by at least one substituent Z²R¹¹, and which cyclic group R⁶ may further be optionally substituted by at least one substituent independently selected from hydroxyl, C₁₋₄ alkyl and phenyl;

R¹¹ represents carboxyl, C₁₋₆ alkylsulphonylaminocarbonyl or C(O)NHOH;

Y¹ represents a bond;

Z² represents a bond, O(CH₂)₁₋₆, NR⁷(CH₂)₁₋₆ or a C₁₋₆ alkylene; and

R⁷ represents hydrogen or a C₁₋₄ alkyl group.

Pharmaceutically acceptable salts of a compound of formula (I) include, but are not limited to, base salts such as an alkali metal salt for example sodium or potassium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. In another aspect, where the compound is sufficiently basic, suitable salts include acid addition salts such as methanesulphonate, fumarate, hydrochloride, hydrobromide, citrate, maleate and salts formed with phosphoric and sulphuric acid. There may be more than one cation or anion depending on the number of charged functions and the valency of the cations or anions. A preferred pharmaceutically acceptable salt is a hydrochloride or sodium salt.

An in vivo hydrolysable (or cleavable) ester of a compound of formula (I) that contains a carboxyl or a hydroxyl group is, for example, a pharmaceutically acceptable ester which is hydrolysed in the human or animal body to produce the parent acid or alcohol. Suitable pharmaceutically acceptable esters for carboxyl include C₁₋₄alkyl esters, for example, methyl; C₁₋₆alkoxymethyl esters, for example, methoxymethyl; C₁₋₆alkanoyloxymethyl esters, for example, pivaloyloxymethyl; phthalidyl esters; C₃₋₈cycloalkoxycarbonyloxyC₁₋₆alkyl esters, for example, 1-cyclohexylcarbonyloxyethyl; 1,3-dioxolen-2-onylmethyl esters, for example, 5-methyl-1,3-dioxolen-2-onylmethyl; and C₁₋₆alkoxycarbonyloxyethyl esters, for example, 1-methoxycarbonyloxyethyl; and may be formed at any carboxyl group in the compounds of this invention. An in vivo hydrolysable (or cleavable) ester of a compound of formula (I) that contains a hydroxyl group includes inorganic esters such as phosphate esters (including phosphoramidic cyclic esters) and α-acyloxyalkyl ethers and related compounds which as a result of the in vivo hydrolysis of the ester breakdown to give the parent hydroxyl group(s). Examples of α-acyloxyalkyl ethers include acetoxymethoxy and 2,2-dimethylpropionyloxy-methoxy. A selection of in vivo hydrolysable ester forming groups for hydroxyl include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

In an embodiment of the invention, the compound of formula (I) is selected from
1-[6-Chloro-5-[(tricyclo[3.3.1.1^{3,7}]dec-1-ylacetyl)amino]-2-quinolinyl]-4-piperidinecarboxylic acid,
1-[6-Chloro-5-[[(tricyclo[3.3.1.1^{3,7}]dec-1-ylmethyl)amino]carbonyl]-2-quinolinyl]-D-proline,
1-[6-Chloro-5-[[(tricyclo[3.3.1.1^{3,7}]dec-1-ylmethyl)amino]carbonyl]-2-quinolinyl]-(3R)-3-piperidinecarboxylic acid,
6-Chloro-2-[4-(1,5-dihydro-5-oxo-4H-1,2,4-triazol-4-yl)-1-piperidinyl]-N-(tricyclo[3.3.1.1^{3,7}]dec-1-ylmethyl)-5-quinolinecarboxamide,
4-[6-Chloro-5-[[(tricyclo[3.3.1.1^{3,7}]dec-1-ylmethyl)amino]carbonyl]-2-quinolinyl]-1-piperazineacetic acid,
6-Chloro-2-[(3S-3-[[2-(2H-tetrazol-5-yl)ethyl]amino]-1-pyrrolidinyl]-N-(tricyclo[3.3.1.1^{3,7}]dec-1-ylmethyl)-5-quinolinecarboxamide,
1-[6-Chloro-5-[[(tricyclo[3.3.1.1^{3,7}]dec-1-ylmethyl)amino]carbonyl]-2-quinolinyl]-4-piperidinecarboxylic acid,
1-[6-Chloro-5-[[(tricyclo[3.3.1.1^{3,7}]dec-1-ylmethyl)amino]carbonyl]-2-quinolinyl]-4-piperidineacetic acid,
6-Chloro-2-[4-(2H-tetrazol-5-yl)butyl]-N-(tricyclo[3.3.1.1^{3,7}]dec-1-ylmethyl)-5-quinolinecarboxamide,
6-Chloro-2-[4-(4,5-dihydro-5-oxo-1,2,4-oxadiazol-3-yl)butyl]-N-(tricyclo[3.3.1.1^{3,7}]dec-1-ylmethyl)-5-quinolinecarboxamide,
N-[(3S)-1-[6-Chloro-5-[[(tricyclo[3.3.1.1^{3,7}]dec-1-ylmethyl)amino]carbonyl]-2-quinolinyl]-3-pyrrolidinyl]-β-alanine,
N-[(3S)-1-[6-Chloro-5-[[(tricyclo[3.3.1.1^{3,7}]dec-1-ylmethyl)amino]carbonyl]-2-quinolinyl]-3-piperidinyl]-β-alanine,
6-Chloro-2-[(3S)-3-[[2-(2H-tetrazol-5-yl)ethyl]amino]-1-piperidinyl]-N-(tricyclo[3.3.1.1^{3,7}]dec-1-ylmethyl)-5-quinolinecarboxamide,
6-Chloro-2-[(3S)-3-[methyl[2-(2H-tetrazol-5-yl)ethyl]amino]-1-pyrrolidinyl]-N-(tricyclo[3.3.1.1^{3,7}]dec-1-ylmethyl)-5-quinolinecarboxamide,
4-[6-Chloro-5-[[(2-tricyclo[3.3.1.1^{3,7}]dec-1-ylethyl)amino]carbonyl]-2-quinolinyl]-1-piperazinepropanoic acid,
1-[6-Chloro-5-[[(tricyclo[3.3.1.1^{3,7}]dec-1-ylmethyl)amino]carbonyl]-2-quinolinyl]-4-hydroxy-4-piperidinecarboxylic acid,
1-[6-Chloro-5-[[(tricyclo[3.3.1.1^{3,7}]dec-1-ylmethyl)amino]carbonyl]-2-quinolinyl]-4-phenyl-4-piperidinecarboxylic acid,
(1R,5S)-3-[6-Chloro-5-[[(tricyclo[3.3.1.1^{3,7}]dec-1-ylmethyl)amino]carbonyl]-2-quinolinyl]-3-azabicyclo[3.1.0]hexane-6-carboxylic acid,
6-Chloro-2-[4-[[(methylsulfonyl)amino]carbonyl]-1-piperidinyl]-N-(tricyclo[3.3.1.1^{3,7}]dec-1-ylmethyl)-5-quinolinecarboxamide,
6-Chloro-2-[4-[(hydroxyamino)carbonyl]-1-piperidinyl]-N-(tricyclo[3.3.1.1^{3,7}]dec-1-ylmethyl)-5-quinolinecarboxamide,
6-Chloro-2-[4-(1H-1,2,4-triazol-3-ylsulfonyl)-1-piperidinyl]-N-(tricyclo[3.3.1.1^{3,7}]dec-1-ylmethyl)-5-quinolinecarboxamide,
2-[6-Chloro-5-[[(tricyclo[3.3.1.1^{3,7}]dec-1-ylmethyl)amino]carbonyl]-2-quinolinyl]-benzoic acid,
3-[6-Chloro-5-[[(tricyclo[3.3.1.1^{3,7}]dec-1-ylmethyl)amino]carbonyl]-2-quinolinyl]-benzoic acid,
4-[6-Chloro-5-[[(tricyclo[3.3.1.1^{3,7}]dec-1-ylmethyl)amino]carbonyl]-2-quinolinyl]-benzoic acid,
1-[6-Chloro-5-[[(tricyclo[3.3.1.1^{3,7}]dec-1-ylmethyl)amino]carbonyl]-2-quinolinyl]-4-methyl-4-piperidinecarboxylic acid,
N-[6-Chloro-5-[[(tricyclo[3.3.1.1^{3,7}]dec-1-ylmethyl)amino]carbonyl]-2-quinolinyl]-β-alanine,
5-[6-Chloro-5-[[(tricyclo[3.3.1.1^{3,7}]dec-1-ylmethyl)amino]carbonyl]-2-quinolinyl]-3-pyridinecarboxylic acid,
   or a pharmaceutically acceptable salt or an in vivo hydrolysable ester thereof.

The present invention further provides a process for the preparation of a compound of formula (I) as defined above or a pharmaceutically acceptable salt or an in vivo hydrolysable ester thereof, which comprises either:

(a) reacting a compound of formula (III)

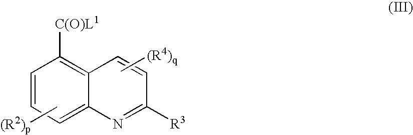

wherein L¹ represents a leaving group (e.g. hydroxyl or halogen) and R², R³, R⁴, p and q are as defined in formula (I), with a compound of formula (IV),

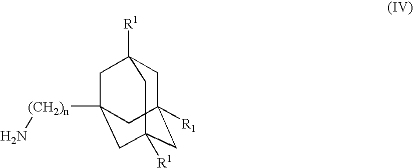

wherein R¹ and n are as defined in formula (I); or

(b) reacting a compound of formula (V)

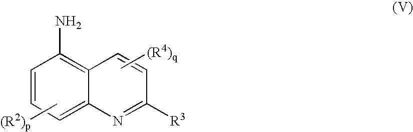

wherein R², R³, R⁴, p and q are as defined in formula (I), with a compound of formula (VI)

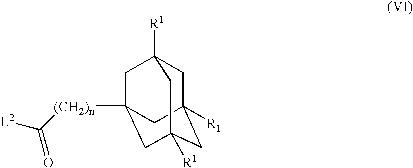

wherein L² represents a leaving group (e.g. hydroxyl or halogen) and R¹ and n are as defined in formula (I); or

(c) when R³ represents a group Y¹R⁶ or Z¹R¹⁰ wherein the atom directly attached to the quinoline group of formula (I) is a nitrogen atom, reacting a compound of formula (VII)

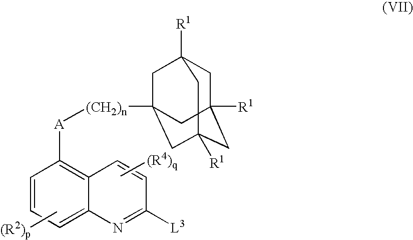

wherein L³ is a leaving group (e.g. halogen, para-toluene sulphonate or methane sulphonate), and all other variables are as defined in relation to formula (I), with a compound of formula (VIII), H—NY^{1″}R^{6″} or formula (IX), H—NZ^{1″}R^{10″} wherein NY^{1″}R^{6″} or NZ^{1″}R^{10″} make up a group of Y¹R⁶ or Z¹R¹⁰ respectively as defined in formula (I); or

(d) when R³ represents a group Y¹R⁶ or Z¹R¹⁰ wherein the group directly attached to the quinoline group of formula (I) is CH₂CH₂, reacting a compound of formula (VII) as defined in (c) above with a compound of formula (X), (XI), (XII) or (XIII)

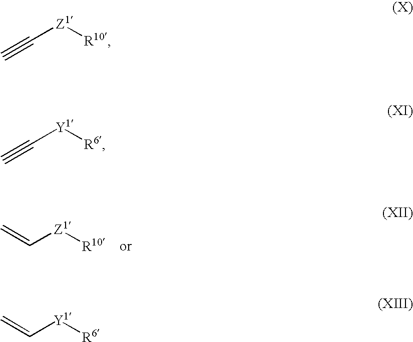

wherein Y^{1′}R^{6′} and Z^{1′}R^{10′} are suitably defined such that reaction of (VII) with (X), (XI), (XII) or (XIII) and subsequent hydrogenation of any resulting alkene or alkyne yields a compound wherein R³ represents a group Y¹R⁶ or Z¹R¹⁰; or

(e) when R³ represents a group Y¹R⁶ or Z¹R¹⁰ wherein the group directly attached to the quinoline group of formula (I) is CH₂CH₂N, reacting a compound of formula (VII) as defined in (c) above with a compound of formula (XIV)

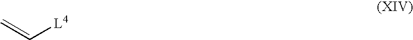

wherein L⁴ is a leaving group (e.g. trialkyltin, dialkylboron or zinc), followed by reaction with a compound of formula (XV), HNY^{1″′}R^{6″′} or (XVI) H Z^{1″′}R^{10″′}, wherein NY^{1″′}R^{6″′} or NZ^{1″′}R^{10″′} make up a group of Y¹R⁶ or Z¹R¹⁰ respectively as defined in formula (I);

(f) when R³ represents a group Y¹R⁶ or Z¹R¹⁰ wherein the group directly attached to the quinoline group of formula (I) is CH₂N, reacting a compound of formula (VII) as defined in (c) above with a compound of formula (XIV) as defined in (e) above, followed by an oxidation reaction and then by reaction with a compound of formula (XV) or (XVI) as defined in (e) above under reductive amination conditions; or

(g) when R³ represents a group Y¹R⁶ or Z¹R¹⁰ wherein the group directly attached to the quinoline group of formula (I) is CH₂CH₂, reacting a compound of formula (VII) as defined in (c) above with a compound of formula (XII) or (XIII) as defined above wherein Y^{1′}R^{6′}, Z^{1′}R^{10′} are suitably defined such that saturation of the alkene and subsequent combination with a compound of formula (VII) yields a compound wherein R³ represents a group Y¹R⁶ or Z¹R¹⁰; or

(h) when R⁸ or R⁹ represent a tetrazole, reacting a compound of formula

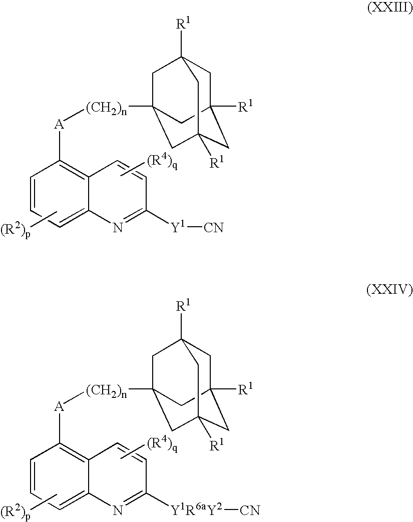

wherein R^{6a} is 4- to 9-membered carbocyclic or heterocyclic ring and all other variables are as defined in relation to formula (I), with a compound of formula PN₃ wherein P is sodium, a trialkylsilyl, an alkyltin or ammonium; or

(i) when R⁸ or R⁹ represent a group of formula (XVII)

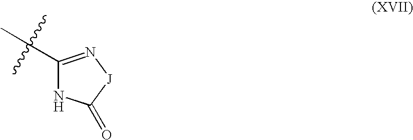

reacting a compound of formula (XXIII) or (XXIV) as defined above in (h) with hydroxylamine, followed by treatment with 1,1′-thiocarbonyldiimidazole and subsequent treatment with silica to yield a compound wherein R⁸ or R⁹ represent a group of formula (XVII) wherein J is S; alternatively reacting a compound of formula (XXVII) or (XXVIII) with hydroxylamine, followed by treatment with a chloroformate to yield a compound wherein R⁸ or R⁹ represent a group of formula (XVII) wherein J is O; or

(j) when R⁸ or R⁹ represent a group of formula (XVIII)

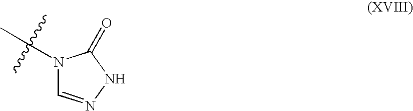

reacting a compound of formula

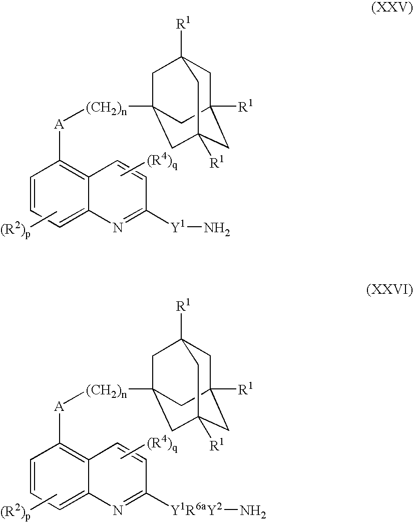

wherein R^{6a} is 4- to 9-membered carbocyclic or heterocyclic ring and all other variables are as defined in relation to formula (I), with phosgene or a phosgene equivalent followed by treatment with formyl hydrazine and subsequent treatment with base; or

(k) when R⁸ or R⁹ represent a group of formula (XIX)

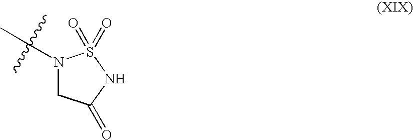

reacting a compound of formula (XXV) or (XXVI) as defined above in (j) with ethyl chloroacetate, followed by reaction with (chlorosulfonyl)-carbamic acid, 1,1-dimethylethyl ester and subsequent treatment with acid and base to yield a compound wherein R⁸ or R⁹ represent a group of formula (XIX); or

(l) when R₃ represents a group Y¹R⁶ wherein Y¹ is a bond and R⁶ is an aromatic carbocyclic or heterocyclic ring substituted by carboxyl, reacting a compound of formula (VII) as defined in (c) above with a compound of formula (XXVII)

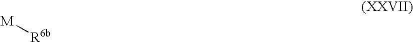

wherein M represents an an organoboron group such as B(OH)₂, B(OⁱPr)₂, BEt₂ or a boronic acid pinacol cyclic ester and R^{6b} represents an aromatic carbocyclic or heterocyclic ring substituted by carboxyl or CO₂C₁₋₆alkyl, optionally followed by reaction with a base.

(m) when R₃ represents a group Y¹R⁶ wherein Y¹ is a bond and R⁶ is an aromatic carbocyclic or heterocyclic ring substituted by carboxyl, reacting a compound of formula (VII) as defined in (c) above with a compound of formula (XXVIII)

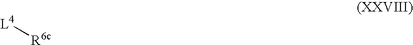

wherein L⁴ represents a leaving group (e.g. halogen) and R^{6c} represents an aromatic carbocyclic or heterocyclic ring substituted by carboxyl, in the presence of a diboron compound such as 4,4,4′,4′,5,5,5′,5′-octamethyl-2,2′-bi-1,3,2-dioxaborolane;

and optionally after (a), (b), (c), (d), (e), (f), (g), (h), (i), (j), (k), (l) or (m) carrying out one or more of the following:
converting the compound obtained to a further compound of the invention
forming a pharmaceutically acceptable salt of the compound
forming an in vivo hydrolysable ester of the compound.

In processes (a) and (b) the coupling reaction is conveniently carried out in an organic solvent such as dichloromethane, N,N-dimethylformamide or 1-methyl-2-pyrrolidinone.

If L¹ or L² represent a hydroxyl group, it may be necessary or desirable to use a coupling agent such as bromo-tris-pyrrolidino-phosphonium hexafluorophosphate (PyBroP). If L¹ or L² are chloride, such compounds may be conveniently prepared by treatment of the corresponding carboxylic acid derivative under standard conditions (such as thionyl chloride in dichloromethane with additional N,N-dimethylformamide) and used in a solvent such as acetone or dichloromethane with a suitable base such as potassium carbonate or triethylamine.

In process (c) the reaction may be performed in an organic solvent such as acetonitrile, N,N-dimethylformamide or 1-methyl-2-pyrrolidinone, and in the presence of a suitable base such as sodium hydride, triethylamine or potassium carbonate at a temperature in the range from, e.g. 50° C. to 150° C., in particular from 80° C. to 120° C., either in a microwave or conventional thermal conditions.

In process (d), the compound of formula (VII) may be conveniently reacted with a compound of formula (X), (XI), (XII) or (XIII) in an organic solvent such as acetonitrile, e.g. at ambient temperature (20° C.), in the presence of catalytic bistriphenylphosphine dichloride palladium(0), copper (I) iodide and a base (e.g. triethylamine). The subsequent hydrogenation reaction may use hydrogen gas with a catalyst such as 5% rhodium on carbon in a solvent, for example, ethyl acetate or ethanol, and at a pressure of 3 bar.

In process (e), the reaction with the vinyl compound of formula (XIV) may conveniently be carried out in a solvent such as N,N-dimethylformamide and in the presence of catalytic dichlorobis(triphenylphosphine)palladium, at elevated temperature, e.g. at about 70° C. The subsequent addition reaction with the resultant compound may be performed under acidic or basic conditions, for example, in acetic acid in a solvent such as methanol or isopropanol at elevated temperature, e.g. at about 100° C.

In process (f), the reaction of the vinyl compound of formula (XIV) may be performed by procedures analogous to those outlined in the previous paragraph on process (e). The subsequent oxidation reaction may be carried out under standard conditions, for example, by using ozone followed by treatment with dimethylsulfide or triphenylphosphine in a suitable solvent such as dichloromethane, or, by using osmium tetroxide and sodium periodate in a suitable solvent such as 1,4-dioxane and water. The reductive amination step may be conveniently carried out in the presence of a reducing agent such as sodium cyanoborohydride, triacetoxyborohydride or sodium borohydride, in a polar solvent such as methanol, ethanol or dichloromethane either alone or in combination with acetic acid.

In process (g), the compound of formula (XII) or (XIII) is pre-treated by reaction with a hydroborating reagent (such as 9-borabicyclo[3.3.1]nonane or catecholborane) in a solvent (such as diethyl ether or tetrahydrofuran) at a temperature in the range from 0° C. to 80° C. (in particular from 60° C. to 70° C.) for about 2 to 3 hours, then cooling the reaction mixture to room temperature and adding a solution of a base (such as sodium hydroxide in water or tri-potassium orthophosphate in water) followed by a solution of the compound of formula (VII) in a solvent (such as N,N-dimethylformamide) and a palladium catalyst (such as tetrakis(triphenylphosphine)palladium(II)). The resulting reaction mixture is stirred at a temperature in the range from 25° C. to 90° C. (particularly from 60° C. to 70° C.) for about 2 to 24 hours to yield the desired compounds of formula (I).

In process (h), the compound of formula (XXIII) or (XXIV) is treated with a compound of the formula PN₃ in a solvent (such as toluene, N,N-dimethylformamide or 1-methyl-2-pyrrolidinone) optionally in the presence of catalyst (such as dibutyltin oxide) at a temperature in the range from 70° C. to 120° C.

In process (i), when in the group of formula (XVII) J=O, a compound of formula (XXIII) or (XXIV) may be treated with hydroxylamine in a suitable solvent (such as methanol or ethanol) at a temperature in the range from 70° C. to 130° C. The resulting intermediate is treated with a suitable chloroformate (such as 2-ethylhexylchloroformate) in a suitable solvent (such as dichloromethane) and heated at a temperature in the range from 70° C. to 150° C. to give the desired compounds of the formula (I). Alternatively, when J=S, treatment of the hydroxylamine adduct with 1,1′-thiocarbonyldiimidazole in a suitable solvent (such as tetrahydrofuran) and addition of silica yields the desired compounds of the formula (I).

In process (j), the compound of formula (XXV) or (XXVI) is treated with phosgene or a phosgene equivalent (such as triphosgene or carbonyldiimidazole) in a suitable solvent (such as dichloromethane) with a suitable base (such as triethylamine). The resulting compound is further treated with formyl hydrazine and this subsequently with a base (such, as potassium hydroxide) in a suitable solvent (such as methanol) at a temperature in the range from 50° C. to 130° C. to give the desired compounds of the formula (I).

In process (k), the compound of formula (XXV) or (XXVI) as defined above in (j) is treated with ethyl chloroacetate in a suitable solvent (such as acetonitrile) with a suitable base (such as triethylamine) at a temperature in the range from 50° C. to 130° C. Treatment of this adduct with (chlorosulfonyl)-carbamic acid, 1,1-dimethylethyl ester in a suitable solvent (such as dichloromethane) and subsequent treatment with a suitable acid (such as trifluoroacetic acid) and a suitable base (such as sodium methoxide) to give the desired compounds of the formula (I).

In process (l), the coupling reaction is conveniently carried out in the presence of a catalyst such as tetrakis(triphenylphosphine)palladium(0), palladium(II) chloride, palladium(II) bromide, dichlorobis(triphenylphosphine)palladium(II), nickel(II) chloride, nickel(II) bromide or bis(triphenylphosphine)nickel(II) chloride, in the presence of a suitable solvent such as tetrahydrofuran, 1,4-dioxane, 1,2-dimethoxyethane, benzene, toluene, xylene, methanol, ethanol or water. The reaction is preferably conducted in the presence of a suitable base such as sodium carbonate or potassium carbonate, pyridine, 4-dimethylaminopyridine, triethylamine or morpholine, and at a temperature in the range 10 to 250° C., preferably in the range 60 to 120° C., either in a microwave or under conventional thermal conditions. Where R^{6b} represents an aromatic carbocyclic or heterocyclic ring substituted CO₂C₁₋₆alkyl, the ester may be converted to carboxyl by reaction with a base such as sodium hydroxide in a solvent such as methanol or water, at a temperature in the range 0 to 100° C.

In process (m), the reaction is conveniently carried out in the presence of a catalyst such as tetrakis(triphenylphosphine)palladium(0), in the presence of a suitable solvent such as tetrahydrofuran or water. The reaction is preferably conducted in the presence of a suitable base such as sodium carbonate, and at a temperature in the range 10 to 250° C., preferably in the range 60 to 150° C., either in a microwave or conventional thermal conditions.

Compounds of formulae (III), (IV), (V), (VI), (X), (XI), (XII), (XIII), (XIV), (XV), (XVI), (XXIII), (XXIV), (XXV), (XXVI), (XXVII) and (XXVIII) are either commercially available, are known in the literature or may be prepared using known techniques.

Examples of preparation methods for certain of these compounds are given hereinafter in the examples. Other examples can be prepared by analogous methods. In particular, compounds of formula (VII) can be prepared by either

(i) reacting a compound of formula (XX)

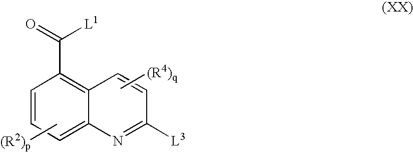

wherein L¹ represents a leaving group (e.g. hydroxyl or halogen) and R², R⁴, p, q and L³ are as defined in formula (I), with a compound of formula (IV) as defined in (a) above;

(ii) reacting a compound of formula (XXI)

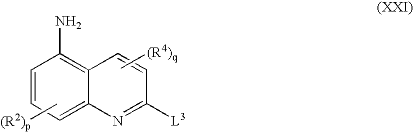

wherein R², R⁴, L³, p and q are as defined in formula (I), with a compound of formula (VI) as defined in (b) above; or

(iii) reacting a compound of formula (XXII)

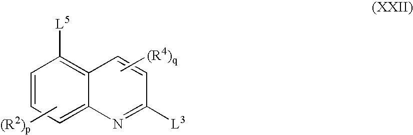

with a suitable amine of formula (IV) as defined above in (a), wherein L⁵ is a halogen (e.g. bromine or iodine) and all other variables are as defined in relation to formula (I) with a suitable source of carbon monoxide and a suitable catalyst.

In (i) and (ii) the coupling reaction is conveniently carried out in an organic solvent such as dichloromethane, N,N-dimethylformamide or 1-methyl-2-pyrrolidinone and reaction conditions analogous to those described above in relation to steps (a) and (b) may be employed;

In (iii) suitable catalysts include palladium catalysts such as dichlorobis(triphenylphosphine)palladium(II) and the reaction may be carried out in an inert solvent such as N-methyl pyrrolidinone, at a temperature between 25° C. and 150° C., preferably 100° C. and under a 1-15 bar pressure of carbon monoxide, preferably 6 bar to give the desired compounds of the formula (VII).

Compounds of formula (XX), (XXI) and (XXII) are either commercially available, are known in the literature or may be prepared using known techniques.

Compounds of formula (XXI) may be prepared by nitration of the corresponding 2-chloroquinoline under standard conditions (such as nitric acid and sulfuric acid at a temperature in the range of 0° C. to 100° C.) followed by subsequent reduction to the corresponding aniline derivative under standard conditions (such as iron powder, acetic acid, hydrochloric acid, water and ethanol at a temperature in the range of 20° C. to 100° C.). Alternatively, a suitable nitro derivative with no substitution at the 2-position may be functionalised to compounds of formula (XXI) by N-oxidation of the quinoline nitrogen under standard conditions (such as peracetic acid in acetic acid at a temperature in the range of 0° C. to 60° C.) and further conversion to its 2-chloro derivative by treatment with a suitable chlorinating agent (such as phosphorus oxychloride at a temperature in the range of 0° C. to 100° C.). 2-Hydroxy compounds may be similarly converted to the 2-chloro derivatives by treatment with similar chlorinating agents.

Compounds of formula (XX) wherein L¹ is OH may be prepared from an appropriately substituted 5-bromo-2-halo-quinoline, such as 5-bromo-2,6-dichloroquinoline, by treatment with a Grignard reagent followed carbon dioxide. Suitable Grignard reagents include isopropylmagnesium chloride and the reaction may be carried out in an inert solvent such as tetrahydrofuran or diethyl ether, at a temperature from −30° C. to 30° C., but preferentially at 0° C. The reaction may be poured onto solid carbon dioxide or more preferably CO₂ gas may be bubbled through the reaction mixture.

The appropriately substituted 5-bromo-2-halo-quinoline may be prepared by bromination of an appropriately substituted 2-haloquinoline such as 2,6-dichloroquinoline. The reaction may be carried by treatment with bromine in the presence of a Lewis acid such as aluminium trichloride at temperatures between −10° C. to 150° C., preferably at 120° C., in the absence of solvent.

The appropriately substituted 5-bromo-quinolines may be prepared by literature methods (J Heterocyclic Chem., 1967, 4, 410, Khimiya Geterotsiklicheskikh Soedinenii, 1988, 8, 1084).

Compounds of formula (I) can be converted into further compounds of formula (I) using standard procedures. For example, compounds of formula (I) in which R² represents a halogen atom may be converted to a corresponding compound of formula (I) in which R² represents a C₁-C₆ alkyl group by reaction with an alkyl Grignard reagent (e.g. methyl magnesium bromide) in the presence of a catalyst such as [1,3-bis(diphenylphosphino)propane]dichloronickel(II) in a solvent such as tetrahydrofuran.

It will be appreciated by those skilled in the art that in the processes of the present invention certain functional groups such as hydroxyl or amino groups in the starting reagents or intermediate compounds may need to be protected by protecting groups. Thus, the preparation of the compounds of formula (I) may involve, at various stages, the addition and removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3rd edition, T. W. Greene and P. G. M. Wuts, Wiley-Interscience (1999).

The compounds of formula (I) above may be converted to a pharmaceutically acceptable salt thereof, such as an acid addition salt, e.g. a hydrochloride, hydrobromide, phosphate, fumarate, maleate, citrate or methanesulphonate, or an alkali metal salt e.g. a sodium or potassium salt, using conventional methods. Other pharmaceutically acceptable salts, as well as in vivo hydrolysable esters, may also be prepared by known methods.

A compound of the invention, or a pharmaceutically acceptable salt or an in vivo hydrolysable ester thereof, can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;

2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;

3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);

4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;

5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;

6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);

7. abdominal: hepatitis, including autoimmune, alcoholic and viral; fibrosis and cirrhosis of the liver; cholecystitis; pancreatitis, both acute and chronic;

8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);

9. allograft rejection: acute and chronic following, for example, transplantation of kidney, heart, liver, lung, bone marrow, skin or cornea or following blood transfusion; or chronic graft versus host disease;

10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;

11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;

12. other disorders with an inflammatory or immunological component; including acquired immune deficiency syndrome (AIDS), leprosy, Sezary syndrome, and paraneoplastic syndromes;

13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;

14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,

15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Accordingly, the present invention provides a compound of formula (I), or a pharmaceutically acceptable salt or an in vivo hydrolysable ester thereof, as hereinbefore defined for use in therapy.

In another aspect, the invention provides the use of a compound of formula (I), or a pharmaceutically acceptable salt or an in vivo hydrolysable ester thereof, as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

The invention further provides a method of effecting immunosuppression (e.g. in the treatment of rheumatoid arthritis, osteoarthritis, irritable bowel disease, atherosclerosis or psoriasis) which comprises administering a therapeutically effective amount of a compound of formula (I), or a pharmaceutically acceptable salt or an in vivo hydrolysable ester thereof, as hereinbefore defined to a patient.

The invention also provides a method of treating an obstructive airways disease (e.g. asthma or COPD) which comprises administering to a patient a therapeutically effective amount of a compound of formula (I), or a pharmaceutically acceptable salt or an in vivo hydrolysable ester thereof, as hereinbefore defined to a patient.

For the above-mentioned therapeutic uses the dosage administered will, of course, vary with the compound employed, the mode of administration, the treatment desired and the disorder indicated. The daily dosage of the compound of formula (I)/salt (“active ingredient”) may be in the range from 0.001 mg/kg to 30 mg/kg.

The compounds of formula (I) and pharmaceutically acceptable salts or in vivo hydrolysable esters thereof may be used on their own but will generally be administered in the form of a pharmaceutical composition in which the formula (I) compound/salt (“active ingredient”) is in association with a pharmaceutically acceptable adjuvant, diluent or carrier. Depending on the mode of administration, the pharmaceutical composition will preferably comprise from 0.05 to 99% w (per cent by weight), more preferably from 0.10 to 70% w, of active ingredient, and, from 1 to 99.95% w, more preferably from 30 to 99.90% w, of a pharmaceutically acceptable adjuvant, diluent or carrier, all percentages by weight being based on total composition.

Thus, the present invention also provides a pharmaceutical composition comprising a is compound of formula (I), or a pharmaceutically acceptable salt or an in vivo hydrolysable ester thereof, as hereinbefore defined in association with a pharmaceutically acceptable adjuvant, diluent or carrier.

The pharmaceutical composition of the invention may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally.

The invention further relates to combination therapies wherein a compound of the invention, or a pharmaceutically acceptable salt or an in vivo hydrolysable ester thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt or an in vivo hydrolysable ester thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt or an in vivo hydrolysable ester thereof, with a monoclonal antibody targeting B-Lymphocytes (such as CD20 (rituximab), MRA-aIL16R and T-Lymphocytes, CTLA4-Ig, HuMax I1-15).

The present invention still further relates to the combination of a compound of the invention, or a pharmaceutically acceptable salt or an in vivo hydrolysable ester thereof, with a modulator of chemokine receptor function such as an antagonist of CCR1, CCR2, CCR2A, CCR2B, CCR3, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention, or a pharmaceutically acceptable salt or an in vivo hydrolysable ester thereof, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt or an in vivo hydrolysable ester thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY×1005.

The present invention further relates to the combination of a compound of the invention, or a pharmaceutically acceptable salt or an in vivo hydrolysable ester thereof, and a receptor antagonist for leukotrienes (LT) B4, LTC4, LTD4, and LTE4 selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention, or a pharmaceutically acceptable salt or an in vivo hydrolysable ester thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt or an in vivo hydrolysable ester thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt or an in vivo hydrolysable ester thereof, and a proton pump inhibitor (such as omeprazole) or a gastroprotective histamine type 2 receptor antagonist.

The present invention further relates to the combination of a compound of the invention, or a pharmaceutically acceptable salt or an in vivo hydrolysable ester thereof, and an antagonist of the histamine type 4 receptor.

The present invention still further relates to the combination of a compound of the invention, or a pharmaceutically acceptable salt or an in vivo hydrolysable ester thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt or an in vivo hydrolysable ester thereof, and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt or an in vivo hydrolysable ester thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt or an in vivo hydrolysable ester thereof, and a chromone, such as sodium cromoglycate or nedocromil sodium.

The present invention still further relates to the combination of a compound of the invention, or a pharmaceutically acceptable salt or an in vivo hydrolysable ester thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt or an in vivo hydrolysable ester thereof, with an agent that modulates a nuclear hormone receptor such as PPARs.

The present invention still further relates to the combination of a compound of the invention, or a pharmaceutically acceptable salt or an in vivo hydrolysable ester thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt or an in vivo hydrolysable ester thereof, and another systemic or topically-applied anti-inflammatory agent, such as thalidomide or a derivative thereof, a retinoid, dithranol or calcipotriol.

The invention still further relates to the combination of a compound of the invention, or a pharmaceutically acceptable salt or an in vivo hydrolysable ester thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt or an in vivo hydrolysable ester thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt or an in vivo hydrolysable ester thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt or an in vivo hydrolysable ester thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt or an in vivo hydrolysable ester thereof, and an agent for the treatment of acute or chronic pain, such as a centrally or peripherally-acting analgesic (for example an opioid or derivative thereof), carbamazepine, phenytoin, sodium valproate, amitryptiline or other anti-depressant agents, paracetamol, or a non-steroidal anti-inflammatory agent.

The present invention further relates to the combination of a compound of the invention, or a pharmaceutically acceptable salt or an in vivo hydrolysable ester thereof, together with a parenterally or topically-applied (including inhaled) local anaesthetic agent such as lignocaine or a derivative thereof.

A compound of the present invention, or a pharmaceutically acceptable salt or an in vivo hydrolysable ester thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt or an in vivo hydrolysable ester thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B1.- or B2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK1 or NK3 receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2X7; or (xxvii) inhibitor of transcription factor activation such as NFkB, API, or STATS.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);

(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;

(iii) an agent which inhibits cancer cell invasion (for example a metalloproteinase inhibitor like marimastat or an inhibitor of urokinase plasminogen activator receptor function);

(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD 1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;

(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);

(vi) a vascular damaging agent such as combretastatin A4, or a compound disclosed in WO 99/02166, WO 00/40529, WO 00/41669, WO 01/92224, WO 02/04434 or WO 02/08213;

(vii) an agent used in antisense therapy, for example one directed to one of the targets listed above, such as ISIS 2503, an anti-ras antisense;

(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or

(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The present invention will now be further explained by reference to the following illustrative examples. In the examples the NMR spectra were measured on a Varian Unity spectrometer at a proton frequency of either 300 or 400 MHz. The MS spectra were measured on either an Agilent 1100 MSD G1946D spectrometer or a Hewlett Packard HP1100 MSD G1946A spectrometer. Preparative HPLC separations were performed using a Waters Symmetry® or Xterra® column using 0.1% aqueous trifluoroacetic acid: acetonitrile, 0.1% aqueous ammonia: acetonitrile or 0.1% ammonium acetate: acetonitrile as the eluent. SCX and NH₂ resin were obtained from Varian Incorporated.

### EXAMPLE 1

### 1-[6-Chloro-5-[(tricyclo[3.3.1.1^{3,7}]dec-1-ylacetyl)amino]-2-quinolinyl]-4-piperidinecarboxylic acid

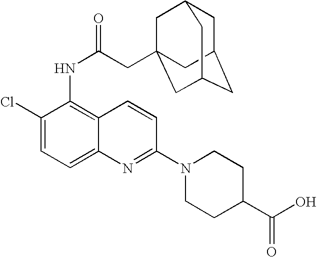

### a) 1-[6-Chloro-5-[(tricyclo[3.3.1.1^{3,7}]dec-1-ylacetyl)amino]-2-quinolinyl]-4-piperidinecarboxylic acid, ethyl ester

To N-(2,6-dichloro-5-quinolinyl)-tricyclo[3.3.1.1^{3,7}]decane-1-acetamide (prepared as described in WO2003080579) (150 mg) and triethylamine (500 μL) in acetonitrile (2 mL) was added 4-piperidinecarboxylic acid, ethyl ester (500 μL). The mixture was heated in a microwave for 40 minutes at 120° C. and then cooled to room temperature. The resulting precipitate was removed by filtration and purified by chromatography (SiO₂, methanol:dichloromethane 5:95 as eluant) to afford the subtitled compound as a solid (100 mg).

MS: APCI(+ve) 510.3/512.3 (M+H⁺).

### b) 1-[6-Chloro-5-[(tricyclo[3.3.1.1^{3,7}]dec-1-ylacetyl)amino]-2-quinolinyl]-4-piperidinecarboxylic acid

Potassium hydroxide (100 mg) was added to a solution of 1-[6-chloro-5-[(tricyclo[3.3.1.1^{3,7}]dec-1-ylacetyl)amino]-2-quinolinyl]-4-piperidinecarboxylic acid, ethyl ester (Example 1(a)) in methanol (2 mL) and water (1 mL). The mixture was stirred at 80° C. for 30 minutes. The reaction mixture was acidified to pH5 with aqueous 2M hydrochloric acid. The resulting precipitate was removed by filtration and purified by chromatography (SiO₂, methanol:dichloromethane:acetic acid 5:95:1 as eluant). Further purification by HPLC (Symmetry—0.1% aqueous ammonium acetate/acetonitrile) afforded the title product (25 mg).

¹H NMR (400 MHz, d₆-DMSO) δ 9.70 (1H, s), 7.88 (1H, d), 7.63-7.42 (2H, m), 7.32 (1H, d), 4.48-4.32 (2H, m), 3.18-2.97 (2H, m), 2.20 (2H, s), 2.05-1.81 (8H, m), 1.79-1.45 (12H, m).MS: APCI(−ve) 480.2/482.2 (M−H⁺).

m.p. 240-241° C.

### EXAMPLE 2

### 1-[6-Chloro-5-[[(tricyclo[3.3.1.1^{3,7}]dec-1-ylmethyl)amino]carbonyl]-2-quinolinyl]-D-proline

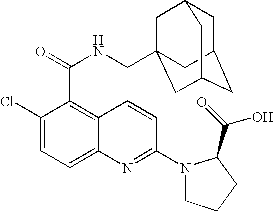

To 2,6-dichloro-N-(tricyclo[3.3.1.1^{3,7}]dec-1-ylnethyl)-5-quinolinecarboxamide (prepared as described in WO2003080579) (250 mg) and triethylamine (269 μL) in acetonitrile (4 mL) was added D-proline 1,1-dimethylethyl ester (330 mg). The mixture was heated in a microwave for 90 minutes at 140° C. and then cooled to room temperature. The solvent was removed from the reaction followed by the addition of water and acetic acid (1 mL) which was subsequently extracted three times with dichloromethane. The combined organics were washed once with water, once with brine followed by the addition of trifluoroacetic acid (1 mL) and stirred for 12 h. The solvent was removed followed by the addition of methanol and placed down a Varian NH₂ cartridge, washed with methanol and eluted using 10% acetic acid in methanol. Removal of solvent afforded the title compound (265 mg).

¹H NMR (300 MHz, d₆-DMSO) δ 8.54 (1H, t), 7.79 (1H, d), 7.54-7.45 (2H, m), 7.00 (1H, s), 4.62-4.55 (1H, m), 3.72-3.54 (2H, m), 3.04 (2H, d), 2.35-2.22 (1H, m), 2.12-1.92 (6H, m), 1.73-1.53 (12H, m).

MS: APCI(+ve) 468 (M+H⁺).

m.p. 157-160° C.

### EXAMPLE 3

### 1-[6-Chloro-5-[[(tricyclo[3.3.1.1^{3,7}]dec-1-ylmethyl)amino]carbonyl]-2-quinolinyl]-(3R)-3-piperidinecarboxylic acid

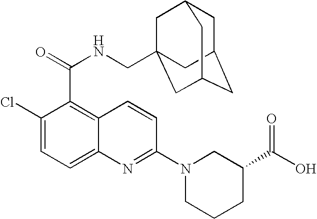

To 2,6-dichloro-N-(tricyclo[3.3.1.1^{3,7}]dec-1-ylmethyl)-5-quinolinecarboxamide (prepared as described in WO2003080579) (250 mg) and triethylamine (536 μL) in acetonitrile (4 mL) was added (3R)-3-piperidinecarboxylic acid ethyl ester L-tartrate (591 mg). The mixture was heated in a microwave for 40 minutes at 140° C. and then cooled to room temperature. Sodium hydroxide solution (2M, 2 mL) and methanol (2 mL) were added to the reaction and heated in a microwave at 70° C. for 30 minutes. The reaction was acidified with hydrochloric acid followed by removal of volatiles, dissolved in dimethylsulphoxide/methanol and purified as described in Example 2. Removal of solvent afforded the title compound (73 mg).

1H NMR (400 MHz, d₆-DMSO) δ 8.54 (1H, t), 7.77 (1H, d), 7.53 (2H, dd), 7.37 (1H, d), 4.55-4.48 (1H, m), 4.27-4.20 (1H, m), 3.22-3.11 (2H, m), 3.03 (2H, d), 2.46-2.35 (1H, m), 2.02-1.93 (4H, m), 1.76-1.54 (14H, m), 1.53-1.39 (1H, m).

MS: APCI(−ve) 480 (M−H⁺).

m.p. 257-262° C.

### EXAMPLE 4

### 6-Chloro-2-[4-(1,5-dihydro-5-oxo-4H-1,2,4-triazol-4-yl)-1-piperidinyl]-N-(tricyclo[3.3.1.1^{3,7}]dec-1-ylmethyl)-5-quinolinecarboxamide

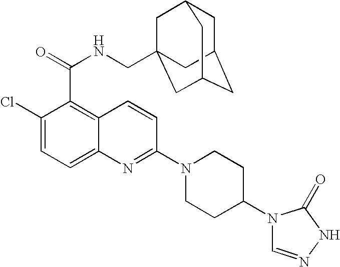

To 2,6-dichloro-N-(tricyclo[3.3.1.1^{3,7}]dec-1-ylmethyl)-5-quinolinecarboxamide (prepared as described in WO2003080579) (250 mg), tert-butylammonium bromide (206 mg) and triethylamine (178 μL) in acetonitrile (4 mL) was added 2,4-dihydro-4-(4-piperidinyl)-3H-1,2,4-triazol-3-one (prepared as described in WO2003080579) (216 mg). The mixture was heated in a microwave for 2.5 h at 140° C. and then cooled to room temperature. The resulting precipitate was collected by filtration, triturated with refluxing methanol, cooled and filtered. The resulting product was taken up in dimethylsulfoxide and absorbed onto SCX, washed with methanol and eluted with 10% ammonia in methanol. The solution was left for 12 h before the precipitate was filtered to afford the title compound (79 mg).

¹H NMR (400 MHz, d₆-DMSO) δ 11.64 (1H, s), 8.55 (1H, t), 7.98-7.96 (1H, m), 7.80 (1H, d), 7.55 (2H, dd), 7.43 (1H, d), 4.71 (2H, d), 4.12-4.02 (1H, m), 3.10-3.00 (4H, m), 1.99-1.89 (5H, m), 1.84-1.72 (2H, m), 1.72-1.54 (12H, m)

MS: APCI(+ve) 521 (M+H⁺).

m.p. 294-295° C.

### EXAMPLE 5

### 4-[6-Chloro-5-[[(tricyclo[3.3.1.1^{3,7}]dec-1-ylmethyl)amino]carbonyl]-2-quinolinyl]-1-piperazineacetic acid

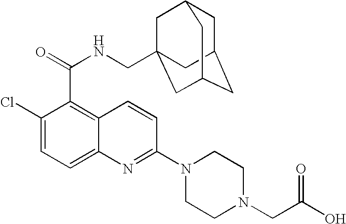

To 2,6-dichloro-N-(tricyclo[3.3.1.1^{3,7}]dec-1-ylmethyl)-5-quinolinecarboxamide (prepared as described in WO2003080579) (250 mg) and triethylamine (179 μL) in acetonitrile (3 mL) was added 1-piperazineacetic acid ethyl ester (332 mg). The mixture was heated in a microwave for 60 minutes at 120° C. and then cooled to room temperature. The reaction was added to 1M sodium hydroxide solution which was then extracted three times with dichloromethane. The combined organics were washed with water, brine and dried. The crude mixture was redissolved in methanol (2 mL) and aqueous sodium hydroxide (2M, 2 mL) added. The mixture was heated in a microwave for 20 minutes at 70° C. after which the reaction was acidified with hydrochloric acid and volatiles removed. The product was triturated with water, filtered and redissolved in methanol. Purification (Varian NH₂ cartridge, eluted with methanol and then 10% acetic acid in methanol) afforded the title compound (78 mg).

¹H NMR (400 MHz, d₆-DMSO) δ 8.55 (1H, t), 7.79 (1H, d), 7.55 (2H, dd), 7.36 (1H, d), 3.76-3.69 (4H, m), 3.19 (2H, s), 3.04 (2H, d), 2.70-2.61 (4H, m), 1.96 (3H, s), 173-1.65 (3H, m), 1.65-1.53 (9H, m).

MS: APCI(+ve) 497 (M+H⁺).

m.p. 179-183° C.

### EXAMPLE 6

### 6-Chloro-2-[(3S)-3-[[2-(2H-tetrazol-5-yl)ethyl]amino]-1-pyrrolidinyl]-N-(tricyclo[3.3.1.1^{3,7}]dec-1-ylmethyl)-5-quinolinecarboxamide

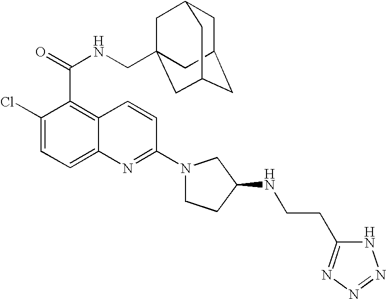

### a) 3-[(3S)-3-Pyrrolidinylamino]-propanenitrile

3-[[(3S)-1-(Phenylmethyl)-3-pyrrolidinyl]amino]-propanenitrile (WO2000075137) (0.6 g), 20% palladium hydroxide on carbon (0.15 g), 1,4-cyclohexadiene (3 mL) and ethanol (2 mL) were loaded into a vial and heated at 100° C. for 90 minutes within a microwave. The reaction mixture was filtered and volatiles removed under vacuum to afford the sub-title compound (0.304 g).

MS: APCI(+ve) 140.3 (M+H⁺).

### b) 6-Chloro-2-[(3S)-3-[(2-cyanoethyl)amino]-1-pyrrolidinyl]-N-(tricyclo[3.3.1.1^{3,7}]dec-1-ylmethyl)-5-quinolinecarboxamide

To 2,6-dichloro-N-(tricyclo[3.3.1.1^{3,7}]dec-1-ylmethyl)-5-quinolinecarboxamide (prepared as described in WO2003080579) (250 mg) and triethylamine (267 μL) in acetonitrile (3 mL) was added 3-[(3S)-3-pyrrolidinylamino]-propanenitrile (Example 6 (a)) (267 mg). The mixture was heated in a microwave for 3 h at 130° C. and then cooled to room temperature. Purification by HPLC (Symmetry—0.1% aqueous ammonium acetate/acetonitrile) afforded the sub-title compound (200 mg).

MS: APCI(+ve) 492 (M+H⁺).

### c) 6-Chloro-2-[(3S)-3-[[2-(2H-tetrazol-5-yl)ethyl]amino]-1-pyrrolidinyl]-N-(tricyclo[3.3.1.1^{3,7}]dec-1-ylmethyl)-5-quinolinecarboxamide

To 6-chloro-2-[(3S)-3-[(2-cyanoethyl)amino]-1-pyrrolidinyl]-N-(tricyclo[3.3.1.1^{3,7}]dec-1-ylmethyl)-5-quinolinecarboxamide (Example 6 (b)) (200 mg), was added trimethylsilylazide (162 μL), dibutyltinoxide (10 mg) and toluene (4 mL). The mixture was heated in a microwave for 90 minutes at 110° C. and then cooled to room temperature. Methanol (5 mL) was added to the crude mixture which was then absorbed onto a Varian NH₂ cartridge, washed with methanol and eluted with 10% acetic acid in methanol. The solvent was removed and the solid triturated with acetonitrile to afford the title compound (76 mg).

¹H NMR (400 MHz, d₆-DMSO) δ 8.56 (1H, t), 7.78 (1H, d), 7.54 (2H, dd), 7.01 (1H, d), 3.83-3.76 (1H, m), 3.72-3.63 (2H, m), 3.59-3.47 (2H, m), 3.14-3.08 (2H, m), 3.06-2.98 (4H, m), 2.29-2.19 (1H, m), 2.05-1.94 (1H, m), 1.96 (3H, s), 1.72-1.65 (3H, m), 1.64-1.54 (9H, m).

MS: APCI(+ve) 535 (M+H⁺).

m.p. 174-177° C.

### EXAMPLE 7

### 1-[6-Chloro-5-[[(tricyclo[3.3.1.1^{3,7}]dec-1-ylmethyl)amino]carbonyl]-2-quinolinyl]-4-piperidinecarboxylic acid

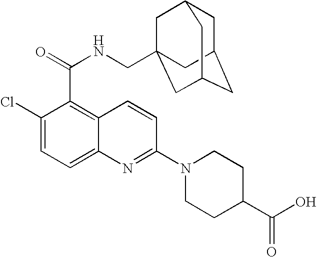

The title compound was prepared as described in Example 5 using 2,6-dichloro-N-(tricyclo[3.3.1.1^{3,7}]dec-1-ylmethyl)-5-quinolinecarboxamide (prepared as described in WO2003080579) (250 mg), triethylamine (179 μL) and 4-piperidinecarboxylic acid ethyl ester (303 mg) in acetonitrile (4 mL) to afford the title compound (33 mg).

1H NMR (400 MHz, d₆-DMSO) δ 8.53 (1H, t), 7.76 (1H, d), 7.52 (2H, dd), 7.35 (1H, d), 4.37 (2H, d), 3.13-3.01 (4H, m), 1.96 (3H, s), 1.90-1.80 (5H, m), 1.75-1.45 (12H, m).

MS: APCI(+ve) 482 (M+H⁺).

m.p. 231-235° C.

### EXAMPLE 8

### 1-[6-Chloro-5-[[(tricyclo[3.3.1.1^{3,7}]dec-1-ylmethyl)amino]carbonyl]-2-quinolinyl]-4-piperidineacetic acid

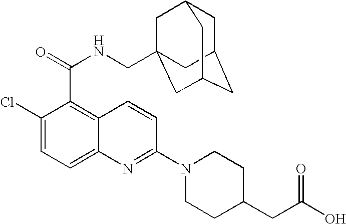

The title compound was prepared as described in Example 5 using 2,6-dichloro-N-(tricyclo[3.3.1.1^{3,7}]dec-1-ylmethyl)-5-quinolinecarboxamide (prepared as described in WO2003080579) (250 mg), triethylamine (179 μL) and 4-piperidineacetic acid ethyl ester (329 mg) in acetonitrile (4 mL). Further purification was achieved by trituration with hot acetonitrile which was filtered to afford the title compound (29 mg).

¹H NMR (400 MHz, d₆-DMSO) δ 8.53 (1H, t), 7.76 (1H, d), 7.52 (2H, dd), 7.35 (1H, d), 4.52 (2H, d), 3.03 (2H, d), 2.94 (2H, t), 2.17 (2H, d), 2.04-1.92 (4H, m), 1.77 (2H, d), 1.72-1.53 (12H, m), 1.25-1.10 (2H, m).

MS: APCI(+ve) 496(M+H⁺).

m.p. 212-214° C.

### EXAMPLE 9

### 6-Chloro-2-[4-(2H-tetrazol-5-yl)butyl]-N-(tricyclo[3.3.1.1^{3,7}]dec-1-ylmethyl)-5-quinolinecarboxamide

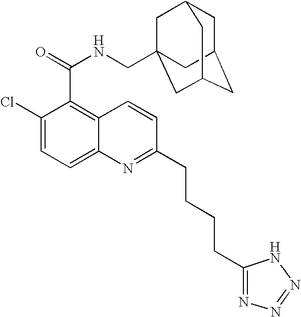

### a) 6-Chloro-2-(4-cyanobutyl)-N-(tricyclo[3.3.1.1^{3,7}]dec-1-ylmethyl)-5-quinolinecarboxamide

2,6-Dichloro-N-(tricyclo[3.3.1.1^{3,7}]dec-1-ylmethyl)-5-quinolinecarboxamide (prepared as described in WO2003080579) (500 mg), tetrakis(triphenylphosphine)palladium (74 mg) and 0.5M cyanobutylzinc bromide in tetrahydrofuran (20 mL) were refluxed under nitrogen for 1 hour. The reaction was cooled to room temperature before the addition of saturated ammonium chloride solution which was subsequently extracted three times with ethyl acetate. The combined organics were washed with water, brine and dried to afford the sub-title compound (652 mg).

MS: APCI(+ve) 436 (M+H⁺).

### b) 6-Chloro-2-[4-(2H-tetrazol-5-yl)butyl]-N-(tricyclo[3.3.1.1^{3,7}]dec-1-ylmethyl)-5-quinolinecarboxamide

The title compound was prepared as described in Example 6 (c) using 6-chloro-2-(4-cyanobutyl)-N-(tricyclo[3.3.1.1^{3,7}]dec-1-ylmethyl)-5-quinolinecarboxamide (Example 9 (a)) (200 mg), trimethylsilylazide (183 μL), dibutyltinoxide (15 mg) and toluene (2 mL). Further purification was achieved by recrystallisation from methanol/acetonitrile to afford the title compound (90 mg).

1H NMR (400 MHz, d₆-DMSO) δ 8.62 (1H, t), 8.02 (1H, d), 7.96 (1H, d), 7.76 (1H, d), 7.56 (1H, d), 3.07 (2H, d), 2.95 (4H, quintet), 1.96 (3H, s), 1.85-1.73 (4H, m), 1.72-1.54 (12H, m).

MS: APCI(+ve) 479 (M+H⁺).

m.p. 221° C.

### EXAMPLE 10

### 6-Chloro-2-[4-(4,5-dihydro-5-oxo-1,2,4-oxadiazol-3-yl)butyl]-N-(tricyclo[3.3.1.1^{3,7}]dec-1-ylmethyl)-5-quinolinecarboxamide

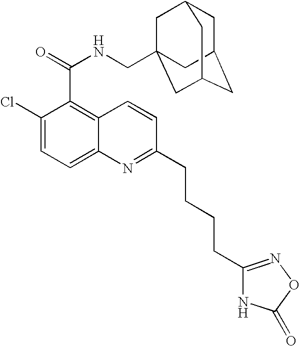

### a) 2-[5-Amino-5-(hydroxyimino)pentyl]-6-chloro-N-(tricyclo[3.3.1.1^{3,7}]dec-1-ylmethyl)-5-quinolinecarboxamide

To 6-chloro-2-(4-cyanobutyl)-N-(tricyclo[3.3.1.1^{3,7}]dec-1-ylmethyl)-5-quinolinecarboxamide (Example 9 (a)) (250 mg) in ethanol (2 mL) was added hydroxylamine (50% w/v solution) (300 μL). The mixture was heated in a microwave for 50 minutes at 90° C. and then cooled to room temperature before removal of volatiles to afford the sub-title compound (280 mg).

MS: APC(+ve) 469 (M+H⁺).

### b) 6-Chloro-2-[4-(4,5-dihydro-5-oxo-1,2,4-oxadiazol-3-yl)butyl]-N-(tricyclo[3.3.1.1^{3,7}]dec-1-ylmethyl)-5-quinolinecarboxamide

To a stirred solution of 2-[5-amino-5-(hydroxyimino)pentyl]-6-chloro-N- (tricyclo[3.3.1.1^{3,7}]dec-1-ylmethyl)-5-quinolinecarboxamide (Example 10 (a)) (180 mg) and pyridine (34 μL) in dichloromethane (5 mL) cooled to 0° C. was added 2-ethylhexyl-chloroformate (75 μL). After 10 minutes the ice bath was removed and stirred for 2 h before volatiles were removed under reduced pressure. The resulting residue was added to iso-hexane and heated in a microwave for 20 minutes at 140° C. The reaction was cooled to room temperature followed by the addition of methanol (5 mL). The resulting solution was absorbed on a Varian NH₂ cartridge, washed with methanol and eluted using 10% acetic acid in methanol. Further purification (SiO₂, methanol:dichloromethane 2:98 as eluant) afforded the title compound (18 mg).

¹H NMR (300 MHz, d₆-DMSO) δ 12.15 (1H, s), 8.62 (1H, t), 8.03 (1H, d), 7.97 (1H, d), 7.76 (1H, d), 7.57 (1H, d), 3.07 (2H, d), 2.95 (2H, t), 2.59-2.51 (2H, m), 1.97 (3H, s), 1.88-1.75 (2H, m), 1.73-1.55 (14H, m).

MS: APCI(+ve) 495 (M+H⁺).

### EXAMPLE 11

### N-[(3S)-1-[6-Chloro-5-[[(tricyclo[3.3.1.1^{3,7}]dec-1-ylmethyl)amino]carbonyl]-2-quinolinyl]-3-pyrrolidinyl]-β-alanine

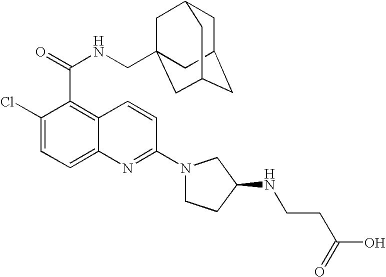

To 6-chloro-2-[(3S)-3-[(2-cyanoethyl)amino]-1-pyrrolidinyl]-N-(tricyclo[3.3.1.1^{3,7}]dec-1-ylmethyl)-5-quinolinecarboxamide (Example 6 (b)) (150 mg) was added potassium hydroxide (68 mg), water (500 μL) and methanol (3 mL). The mixture was heated in a microwave for 3 h at 90° C. before being cooled to room temperature. The reaction was treated with ammonia and volatiles removed under reduced pressure. Purification (SiO₂, methanol:dichloromethane:ammonia 5:94:1 increasing to 10:90:1 as eluant) gave the solid which was further purified using a Varian NH₂ cartridge, washed with methanol and eluted using 10% acetic acid in methanol. The solvent was removed to afford the title compound (71 mg).

¹H NMR (400 MHz, d₆-DMSO) δ 8.55 (1H, t), 7.76 (1H, d), 7.54 (1H, d), 7.49 (1H, d), 6.98 (1H, d), 3.75-3.68 (1H, m), 3.67-3.58 (1H, m), 3.57-3.47 (1H, m), 3.47-3.40 (1H, m), 3.39-3.31 (1H, m), 3.03 (2H, d), 2.84-2.76 (2H, m), 2.30 (2H, t), 2.19-2.09 (1H, m), 1.96 (3H, s), 1.88-1.81 (1H, m), 1.72-1.65 (3H, m), 1.65-1.54 (9H, m).

MS: APCI(+ve) 511 (M+H⁺).

### EXAMPLE 12

### N-[(3S)-1-[6-Chloro-5-[[(tricyclo[3.3.1.1^{3,7}]dec-1-ylmethyl)amino]carbonyl]-2-quinolinyl]-3-piperidinyl]-β-alanine

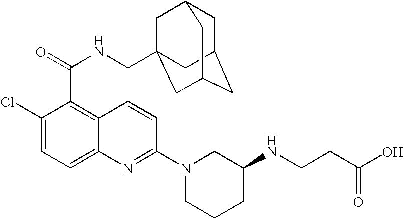

### a) 2-[(3S)-3-Amino-1-piperidinyl]-6-chloro-N-(tricyclo[3.3.1.1^{3,7}]dec-1-ylmethyl)-5-quinolinecarboxamide

To 2,6-dichloro-N-(tricyclo[3.3.1.1^{3,7}]dec-1-ylmethyl)-5-quinolinecarboxamide (prepared as described in WO2003080579) (389 mg) in triethylamine (4 mL) was added (3S)-3-piperidinamine (200 mg). The mixture was heated in a microwave for 60 minutes at 140° C. and then cooled to room temperature before being poured onto water followed by the addition of sodium hydrogen carbonate solution. This solution was extracted with dichloromethane and the combined organics washed with water and brine. Removal of the solvent afforded the sub-title compound (450 mg).

MS: APCI(+ve) 453 (M+H⁺).

### b) 6-Chloro-2-[(3S)-3-[(2-cyanoethyl)amino]-1-piperidinyl]-N-(tricyclo[3.3.1.1^{3,7}]dec-1-ylmethyl)-5-quinolinecarboxamide

2-[(3S)-3-Amino-1-piperidinyl]-6-chloro-N-(tricyclo[3.3.1.1^{3,7}]dec-1-ylmethyl)-5-quinolinecarboxamide (Example 12 (a)) (200 mg) in 2-propenenitrile (1 mL) was heated in a microwave for 40 minutes at 120° C. and then cooled to room temperature before volatiles were removed under reduced pressure. The residue was dissolved in methanol and absorbed onto SCX, washed with methanol and eluted with 10% ammonia in methanol. The solvent was removed to afford the sub-title compound (160 mg).

MS: APCI(+ve) 506 (M+H⁺).

### c) N-[(3S)-1-[6-Chloro-5-[[(tricyclo[3.3.1.1^{3,7}]dec-1-ylmethyl)amino]carbonyl]-2-quinolinyl]-3-piperidinyl]-β-alanine

To 6-chloro-2-[(3S)-3-[(2-cyanoethyl)amino]-1-piperidinyl]-N-(tricyclo[3.3.1.1^{3,7}]dec-1-ylmethyl)-5-quinolinecarboxamide (Example 12 (b)) (130 mg) in methanol (5 mL) was added sodium hydroxide solution (47% w/v) (200 μL). The mixture was heated in a microwave for 120 minutes at 90° C. and then cooled to room temperature before volatiles were removed under reduced pressure. The residue was acidified with hydrochloric and then re-basified with ammonia. The volatiles were removed under reduced pressure and the residue purifed (SiO₂, methanol:dichloromethane:ammonia 6:93:1 increasing to 8:91:1 as eluant). Further purification using SCX resin, washing with methanol and then eluting with 10% ammonia in methanol gave the title compound (13 mg).

¹H NMR (400 MHz, d₆-DMSO) δ 8.54 (1H, t), 7.78 (1H, d), 7.54 (2H, d), 7.38 (1H, d), 4.42 (1H, d), 4.15 (1H, d), 3.23-3.16 (1H, m), 3.13-3.06 (1H, m), 3.04 (2H, d), 3.01-2.95 (2H, m), 2.84-2.77 (1H, m), 2.39 (2H, t), 2.03-1.95 (1H, m), 1.96 (3H, s), 1.81-1.73 (1H, m), 1.72-1.65 (3H, m), 1.65-1.54 (9H, m), 1.52-1.45 (2H, m)

MS: APCI(+ve) 525 (M+H⁺).

m.p. 176-189° C.

### EXAMPLE 13

### 6-Chloro-2-[(3S)-3-[[2-(2H-tetrazol-5-yl)ethyl]amino]-1-piperidinyl]-N-(tricyclo[3.3.1.1^{3,7}]dec-1-ylmethyl)-5-quinolinecarboxamide

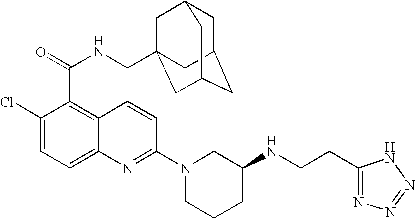

The title compound was prepared and purified as described in Example 6 (c) using 6-chloro-2-[(3S)-3-[(2-cyanoethyl)amino]-1-piperidinyl]-N-(tricyclo[3.3.1.1^{3,7}]dec-1-ylmethyl)-5-quinolinecarboxamide (Example 12 (b)) (200 mg), trimethylsilylazide (117 μL), dibutyltinoxide (23 mg) and toluene (2 mL). Purification (SiO₂, methanol:dichloromethane:ammonia 10:90:1 eluant) gave an oil which was further purified using SCX, washing with methanol and then eluting with 10% ammonia in methanol to afford the title compound (50 mg).

¹H NMR (400 MHz, d₆-DMSO) δ 8.54 (1H, t), 7.80 (1H, d), 7.59-7.52 (2H, m), 7.39 (1H, d), 4.52-4.46 (1H, m), 4.18-4.11 (1H, m), 3.30-3.18 (3H, m), 3.12-3.01 (5H, m), 2.10-2.03 (1H, m), 1.96 (3H, s), 1.87-1.77 (1H, m), 1.73-1.54 (15H, m)

MS: APCI(+ve) 549 (M+H⁺).

m.p. 168-172° C.

### EXAMPLE 14

### 6-Chloro-2-[(3S)-3-[methyl[2-(2H-tetrazol-5-yl)ethyl]amino]-1-pyrrolidinyl]-N-(tricyclo[3.3.1.1^{3,7}]dec-1-ylmethyl)-5-quinolinecarboxamide

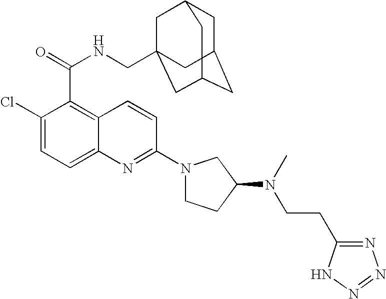

### a) 6-Chloro-2-[(3S)-3-[(2-cyanoethyl)methylamino]-1-pyrrolidinyl]-N-(tricyclo[3.3.1.1^{3,7}]dec-1-ylmethyl)-5-quinolinecarboxamide

To 6-chloro-2-[(3S)-3-[(2-cyanoethyl)amino]-1-pyrrolidinyl]-N-(tricyclo[3.3.1.1^{3,7}dec-1-ylmethyl)-5-quinolinecarboxamide (Example 6 (b)) (360 mg) in methanol (10 mL) was added paraformaldehyde (200 mg) followed by sodium triacetoxyborohydride (300 mg). The reaction was stirred for 2 h before additional paraformaldehyde (200 mg) and sodium triacetoxyborohydride (300 mg) was added. After 2 h the reaction was poured into sodium hydrogen carbonate solution and extracted three times with dichloromethane. The combined organics were washed once with water and dried over magnesium sulphate before being filtered and the solvent removed to afford the sub-title compound (380 mg).

MS: APCI(+ve) 506 (M+H⁺).

### b) 6-Chloro-2-[(3S)-3-[methyl[2-(2H-tetrazol-5-yl)ethyl]amino]-1-pyrrolidinyl]-N-(tricyclo[3.3.1.1^{3,7}]dec-1-ylmethyl)-5-quinolinecarboxamide

The title compound was prepared and purified as described in Example 6 (c) using 6-chloro-2-[(3S)-3-[(2-cyanoethyl)methylamino]-1-pyrrolidinyl]-N-(tricyclo[3.3.1.1^{3,7}]dec-1-ylmethyl)-5-quinolinecarboxamide (Example 14 (a)) (380 mg), trimethylsilylazide (200 μL), dibutyltinoxide (37 mg) and toluene (3 mL). Further purification using SCX, washing with methanol and then eluting with 10% ammonia in methanol afforded the title compound (181 mg).

¹H NMR (300 MHz, d₆-DMSO) δ 8.54 (1H, t), 7.76 (1H, d), 7.57 (1H, d), 7.49 (1H, d), 7.00 (1H, d), 3.91-3.80 (1H, m), 3.79-3.67 (1H, m), 3.50-3.38 (1H, m), 3.28-3.10 (2H, m), 3.03 (2H, d), 2.97-2.88 (2H, m), 2.84-2.75 (2H, m), 2.30 (3H, s), 2.25-2.13 (1H, m), 1.96 (3H, s), 1.88-1.78 (1H, m), 1.73-1.53 (12H, m).

MS: APCI(+ve) 549 (M+H⁺).

### EXAMPLE 15

### 4-[6-Chloro-5-[[(2-tricyclo[3.3.1.1^{3,7}]dec-1-ylethyl)amino]carbonyl]-2-quinolinyl]-1-piperazinepropanoic acid, methyl ester

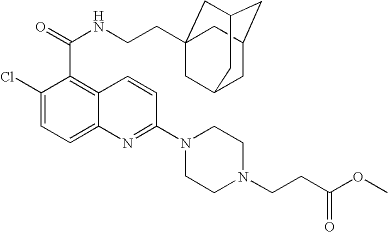

### a) 5-Bromo-2,6-dichloro-quinoline

2,6-Dichloroquinoline (30 g) and aluminium trichloride (60 g) were heated to 120° C. with stirring under a nitrogen atmosphere. Bromine (9.2 mL) was added dropwise over 1 hour and the mixture was then stirred at 120° C. for 1 hour before being cooled to room temperature. A methanol/deionised water mixture (150 mL, 1:1) was then slowly added and the mixture was concentrated in vacuo. Dichloromethane (500 mL) and deionised water (250 mL) were added, the layers were separated and the aqueous fraction was extracted with dichloromethane (2×250 mL). The combined organic extracts were washed with saturated aqueous sodium hydrogen carbonate (250 mL) before being dried, filtered and concentrated. Purification by chromatography (SiO₂, isohexane:dichloromethane 7:3 as eluant) gave the sub-title compound as a solid (27 g).

¹H NMR (400 MHz, CDCl₃) δ 8.53 (1H, d), 7.94 (1H, d), 7.78 (1H, d), 7.50 (1H, d).

MS: APCI(+ve) 276/278/280/282 (M+H⁺).

### b) 2,6-Dichloro-5-quinolinecarboxylic acid

To a stirred solution of 5-bromo-2,6-dichloro-quinoline (Example 15 (a)) (23 g) in tetrahydrofuran (300 mL) at 0° C. was added iso-propylmagnesium chloride (2M in tetrahydrofuran, 42 mL) over 2 hours. CO₂ was bubbled through the solution for 20 minutes and then methanol (20 mL) was added. The mixture was poured into water (500 mL) and extracted with ethyl acetate. The aqueous layer was acidified with hydrochloric acid (2M in water) to pH 2-3 and the resulting solid collected by filtration. The solid was washed with water and dried to afford the sub-titled compound (11.5 g).

¹H NMR (400 MHz, d₆-DMSO) δ 8.29 (1H, d), 8.07 (1H, d), 7.94 (1H, d), 7.74 (1H, d).

### c) 2,6-Dichloro-N-(2-tricyclo[3.3.1.1^{3,7}]dec-1-ylethyl)-5-quinolinecarboxamide

To 2,6-dichloro-5-quinolinecarboxylic acid (Example 15 (b)) (640 mg) in dichloromethane (5 mL) and N,N-dimethylformamide (1 drop) was added oxalyl chloride (500 μL) at 0° C. over 2 minutes. The mixture was allowed to warm to room temperature and heated to 40° C. for 30 minutes. Volatiles were removed under reduced pressure and the mixture redissolved in dichloromethane (5 mL). Tricyclo[3.3.1.1^{3,7}]decane-1-ethanamine (600 mg) and triethylamine (500 μL) in dichloromethane were added dropwise to the mixture which was stirred at room temperature for a further 2 h. The resulting white precipitate was collected by filtration and dried to afford the subtitled compound (410 mg).

MS: APCI(+ve) 402/404 (M+H⁺).

### d) 6-Chloro-2-(1-piperazinyl)-N-(2-tricyclo[3.3.1.1^{3,7}]dec-1-ylethyl)-5-quinolinecarboxamide

To 2,6-dichloro-N-(2-tricyclo[3.3.1.1^{3,7}]dec-1-ylethyl)-5-quinolinecarboxamide (Example 15 (c)) (220 mg) and triethylamine (500 μL) in acetonitrile (4 mL) was added piperazine (500 mg). The reaction was heated in a microwave at 120° C. for 1 h and cooled to room temperature. The mixture was poured into aqueous sodium bicarbonate solution (10 mL) and extracted with dichloromethane (3×20 mL). The combined organic extracts were dried and purified by chromatography (SiO₂, methanol:dichloromethane 1:10 as eluant) to afford the subtitled compound as a solid (200 mg).

MS: APCI(+ve) 453.3 (M+H⁺).

### e) 4-[6-Chloro-5-[[(2-tricyclo[3.3.1.1^{3,7}]dec-1-ylethyl)amino]carbonyl]-2-quinolinyl]-1-piperazinepropanoic acid, methyl ester

To 6-chloro-2-(1-piperazinyl)-N-(2-tricyclo[3.3.1.1^{3,7}]dec-1-ylethyl)-5-quinolinecarboxamide (Example 15 (d)) (200 mg) in dichloromethane (7 mL) and triethylamine (500 μL) at 0° C. was added 2-propenoic acid, methyl ester (500 μL). The mixture was stirred for 1 h and poured into aqueous sodium bicarbonate solution (10 mL) and extracted with dichloromethane (3×20 mL). The combined organic extracts were dried and purified by chromatography (SiO₂, methanol:dichloromethane 1:10 as eluant) to afford the title compound as a solid (120 mg).

¹H NMR (400 MHz, d₆-DMSO) δ 8.54 (1H, t), 7.75 (1H, d), 7.58-7.48 (2H, m), 7.34 (1H, d), 3.68 (4H, m), 3.63 (3H, s), 3.37-3.28 (4H, m), 2.62 (2H, t), 2.56-2.47 (4H, m), 1.95 (3H, m), 1.74-1.59 (6H, m), 1.54 (6H, s), 1.36 (2H, dd).

MS: APCI(+ve) 539.0/541.0 (M+H⁺).

### EXAMPLE 16

### 4-[6-Chloro-5-[[(2-tricyclo[3.3.1.1^{3,7}]dec-1-ylethyl)amino]carbonyl]-2-quinolinyl]-1-piperazinepropanoic acid

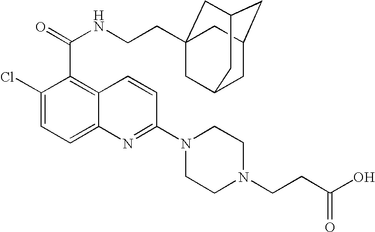

Prepared by the method of Example 1(b) using 4-[6-chloro-5-[[(2-tricyclo[3.3.1.1^{3,7}]dec-1-ylethyl)amino]carbonyl]-2-quinolinyl]-1-piperazinepropanoic acid, methyl ester (Example 15 (e)) and sodium hydroxide (100 mg) in methanol (3 mL) and water (3 mL). The mixture was acidified with aqueous hydrochloric acid, the resulting solid removed by filtration and dried to afford the title compound as a solid (60 mg).

¹H NMR (400 MHz, d₆-DMSO) δ 8.57 (1H, t), 7.81 (1H, d), 7.60 (1H, d), 7.57 (1H, d), 7.40 (1H, d), 3.90 (4H, m), 3.32 (6H, m), 3.05 (2H, m), 2.71 (2H, t), 1.95 (3H, s), 1.76-1.59 (6H, m), 1.54 (6H, s), 1.37 (2H, m).

MS: APCI(+ve) 525.0/527.0 (M+H⁺).

### EXAMPLE 17

### 1-[6-Chloro-5-[[(tricyclo[3.3.1.1^{3,7}]dec-1-ylmethyl)amino]carbonyl]-2-quinolinyl]-4-hydroxy-4-piperidinecarboxylic acid

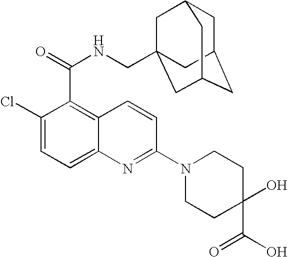

Prepared by the method of Example 3 using 2,6-dichloro-N-(tricyclo[3.3.1.1^{3,7}]dec-1-ylmethyl)-5-quinolinecarboxamide (prepared as described in WO 2003080579) and 4-hydroxy-4-piperidinecarboxylic acid, methyl ester (prepared as described in WO9910999) to afford the title compound (100 mg).

¹H NMR (400 MHz, d₆-DMSO) δ 8.53 (1H, t), 7.78 (1H, d), 7.55 (1H, d), 7.52 (1H, d), 7.38 (1H, d), 4.27 (2H, m), 3.35 (2H, m), 3.03 (2H, d), 1.96 (3H, s), 1.86 (2H, dt), 1.73-1.54 (14H, m).

MS: APCI(+ve) 498/500 (M+H⁺).

m.p. 200-204° C.

### EXAMPLE 18

### 1-[6-Chloro-5-[[(tricyclo[3.3.1.1^{3,7}]dec-1-ylmethyl)amino]carbonyl]-2-quinolinyl]-4-phenyl-4-piperidinecarboxylic acid

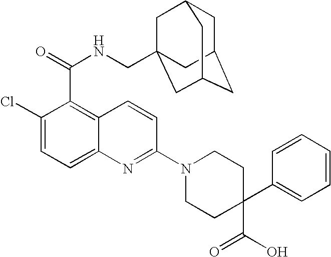

To 2,6-dichloro-N-(tricyclo[3.3.1.1^{3,7}]dec-1-ylmethyl)-5-quinolinecarboxamide (prepared as described in WO2003080579) (250 mg), potassium carbonate (500 mg) in N-methyl pyrrolidinone (4 mL) was added 4-phenyl-4-piperidinecarboxylic acid, p-toluenesulfonate (800 mg). The resulting mixture was heated to 100° C. for 18 h and then cooled to room temperature. The mixture was poured into water and extracted with dichloromethane (3×20 mL). The combined organic extracts were dried and concentrated to yield a solid which was recrystallised from ethyl acetate/methanol to give the title compound as a solid (100 mg).

¹H NMR (400 MHz, d₆-DMSO) δ 8.69 (1H, s), 7.92 (1H, d), 7.77 (1H, m), 7.64 (1H, m), 7.48-7.35 (5H, m), 7.32-7.25 (1H, m), 4.60-4.39 (2H, m), 3.53-3.40 (2H, m), 3.05 (2H, d), 2.59 (2H, d), 2.10-1.92 (2H, m), 1.96 (3H, m), 1.73-1.52 (12H, m).

MS: APCI(−ve) 556/558 (M−H⁺).

m.p. 235-237° C.

### EXAMPLE 19

### (1R,5S)-3-[6-Chloro-5-[[(tricyclo[3.3.1.1^{3,7}]dec-1-ylmethyl)amino]carbonyl]-2-quinolinyl]-3-azabicyclo[3.1.0]hexane-6-carboxylic acid

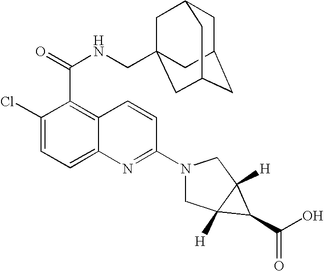

Prepared by the method of Example 3 using 2,6-dichloro-N-(tricyclo[3.3.1.1^{3,7}]dec-1-ylmethyl)-5-quinolinecarboxamide (prepared as described in WO2003080579) (250 mg) and (1R,5S)-3-azabicyclo[3.1.0]hexane-6-carboxylic acid, 1,1-dimethylethyl ester (300 mg). The mixture was evaporated to an oil and treated with trifluoroacetic acid (2.5 mL) in dichloromethane (2.5 mL). After 3 h the mixture was evaporated to an oil and purified by HPLC (Symmetry—0.1% aqueous ammonium acetate/acetonitrile) to afford the title product (120 mg).

¹H NMR (400 MHz, d₆-DMSO) δ 8.54 (1H, t), 7.76 (1H, d), 7.53 (1H, d), 7.51 (1H, d), 6.96 (1H, d), 3.87 (2H, d), 3.56 (2H, d), 3.03 (2H, d), 2.10 (2H, s), 1.96 (3H, s), 1.72-1.58 (6H, m), 1.57 (6H, d), 1.32 (1H, t).

MS: APCI(−ve) 478/480 (M−H⁺).

m.p. 175-180° C.

### EXAMPLE 20

### 6-Chloro-2-[4-[[(methylsulfonyl)amino]carbonyl]-1-piperidinyl]-N-(tricyclo[3.3.1.1^{3,7}]dec-1-ylmethyl)-5-quinolinecarboxamide

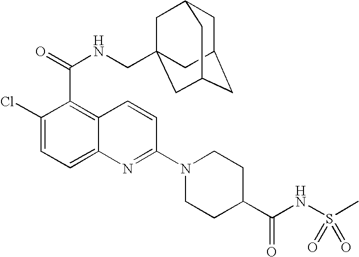

### a) 1-[6-Chloro-5-[[(tricyclo[3.3.1.1^{3,7}]dec-1-ylmethyl)amino]carbonyl]-2- quinolinyl]-4-piperidinecarboxylic acid monosodium salt

The sub-title compound was prepared as described in Example 5. Isolation of the sodium salt was achieved by treatment of the crude product in methanol with sodium hydroxide. After 12 h the resulting solid was filtered and washed with acetonitrile.

MS: APCI(+ve) 482 (M+H⁺).

### b) 6-Chloro-2-[4-[[(methylsulfonyl)amino]carbonyl]-1-piperidinyl]-N-(tricyclo[3.3.1.1^{3,7}]dec-1-ylmethyl)-5-quinolinecarboxamide

To 1-[6-chloro-5-[[(tricyclo[3.3.1.1^{3,7}]dec-1-ylmethyl)amino]carbonyl]-2-quinolinyl]-4-piperidinecarboxylic acid monosodium salt (150 mg) (Example 20 (a)) and HATU (226 mg) in dichloromethane (5 mL) was added sulfamide (57 mg). The reaction was stirred for two hours and then dimethylaminopyridine (37 mg) in tetrahydrofuran (10 mL) added. After 12 hours the solvent was removed under vacuum with the resulting material being taken up in methanol (10 mL) and purified by HPLC (Symmetry—0.1% aqueous ammonium acetate/acetonitrile) to afford the title compound (27 mg).

¹H NMR (300 MHz, d₆-DMSO) δ 8.53 (1H, t), 7.77 (1H, d), 7.58-7.49 (2H, m), 7.36 (1H, d), 4.48 (2H, d), 3.03 (2H, d), 2.99 (3H, s), 2.99-2.93 (2H, m), 2.46-2.37 (1H, m), 1.96 (3H, s), 1.88-1.78 (2H, m), 1.73-1.44 (14H, m)

MS: APCI(+ve) 559 (M+H⁺).

m.p. 170-173° C.

### EXAMPLE 21

### 6-chloro-2-[4-[(hydroxyamino)carbonyl]-1-piperidinyl]-N-(tricyclo[3.3.1.1^{3,7}]dec-1-ylmethyl)-5-quinolinecarboxamide

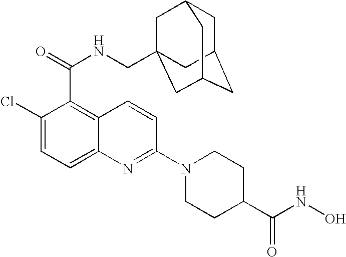

To 1-[6-Chloro-5-[[(tricyclo[3.3.1.1^{3,7}]dec-1-ylmethyl)amino]carbonyl]-2-quinolinyl]-4-piperidinecarboxylic acid monosodium salt (130 mg) (Example 20 (a)), triethylamine (72 μL) and PyBroP (240 mg) in dichloromethane (5 mL) was added hydroxylamine solution (50% w/v in water (100 μL )). The reaction was stirred for 2 h and ethanol (5 mL) added. After 1 h the solvent was removed under vacuum with the resulting material being dissolved in methanol (10 mL) and purified by prep HPLC (Symmetry—0.1% aqueous ammonium acetate/acetonitrile) to afford the title compound (49 mg).

¹H NMR (400 MHz, d₆-DMSO) δ 10.48 (1H, s), 8.72 (1H, s), 8.53 (1H, t), 7.78 (1H, d), 7.54 (2H, q), 7.37 (1H, d), 4.56 (2H, d), 3.04 (2H, d), 2.96 (2H, t), 2.38-2.29 (1H, m), 1.96 (3H, s), 1.74-1.51 (16H, m)

MS: APCI(+ve) 497 (M+H⁺).

m.p. 189-196° C.

### EXAMPLE 22

### 6-Chloro-2-[4-(1H-1,2,4-triazol-3-ylsulfonyl)-1-piperidinyl]-N-(tricyclo[3.3.1.1^{3,7}]dec-1-ylmethyl)-5-quinolinecarboxamide

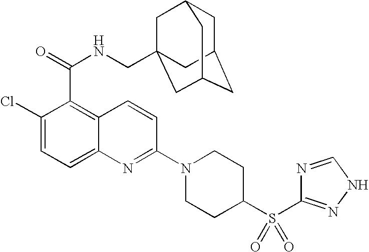

### a) 6-Chloro-2-(4-hydroxy-1-piperidinyl)-N-(tricyclo[3.3.1.1^{3,7}]dec-1-ylmethyl)-5-quinolinecarboxamide

To 2,6-dichloro-N-(tricyclo[3.3.1.1^{3,7}]dec-1-ylmethyl)-5-quinolinecarboxamide (prepared as described in WO2003080579) (500 mg) and triethylamine (536 μL) in acetonitrile (2 mL) was added 4-piperidinol (195 mg). The mixture was heated in a microwave for 4 h at 130° C. and then cooled to room temperature. The resulting precipitate was filtered and washed with acetonitrile to afford the sub-titled compound (543 mg).

MS: APCI(+ve) 454 (M+H⁺).

### b) 6-Chloro-2-[4-[(methylsulfonyl)oxy]-1-piperidinyl]-N-(tricyclo[3.3.1.1^{3,7}]dec-1-ylmethyl)-5-quinolinecarboxamide

To 6-chloro-2-(4-hydroxy-1-piperidinyl)-N-(tricyclo[3.3.1.1^{3,7}]dec-1-ylmethyl)-5-quinolinecarboxamide (543 mg) (Example 22 (a)) and methanesulfonyl chloride (185 μL) in dichloromethane (10 mL) was added triethylamine (333 μL). The reaction was stirred for 12 h, poured onto a saturated solution of sodium hydrogen carbonate which was subsequently extracted with dichloromethane. The combined organics were washed with water, brine and the solvent removed to afford the sub-titled compound (770 mg).

MS: APCI(+ve) 532 (M+H⁺).

### c) 6-Chloro-2-[4-(1H-1,2,4-triazol-3-ylthio)-1-piperidinyl]-N-(tricyclo[3.3.1.1^{3,7}]dec-1-ylmethyl)-5-quinolinecarboxamide

To 6-chloro-2-[4-[(methylsulfonyl)oxy]-1-piperidinyl]-N-(tricyclo[3.3.1.1^{3,7}]dec-1-ylmethyl)-5-quinolinecarboxamide (200 mg) (Example 22 (b)) and potassium carbonate (104 mg) in acetone (2 mL) and water (0.3 mL) was added 1H-1,2,4-triazole-3-thiol (57 mg). The mixture was heated in a microwave for 40 minutes at 120° C. and then cooled to room temperature. The solvent was removed under vacuum and the reaction worked up as described in (Example 22 (b)) to afford the sub-titled compound (220 mg).

MS: APCI(+ve) 538/540 (M+H⁺).

### d) 6-Chloro-2-[4-(1H-1,2,4-triazol-3-ylsulfonyl)-1-piperidinyl]-N-(tricyclo[3.3.1.1^{3,7}]dec-1-ylmethyl)-5-quinolinecarboxamide

Sodium tungstate (3 mg) was added to 6-chloro-2-[4-(1H-1,2,4-triazol-3-ylthio)-1-piperidinyl]-N-(tricyclo[3.3.1.1^{3,7}]dec-1-ylmethyl)-5-quinolinecarboxamide (220 mg) (Example 22 (c)) dissolved in acetonitrile (30 mL) and water (3 mL). After 5 minutes hydrogen peroxide (35% solution) (300 μL) was added and stirred for 12 h whereupon additional hydrogen peroxide (300 μL) was added. After 12 h the volatiles were partially removed under vacuum and the residue purified by HPLC (Symmetry—0.1% aqueous ammonium acetate/acetonitrile) to afford the title compound (58 mg).

¹H NMR (400 MHz, d₆-DMSO) δ 8.53 (1H, t), 8.07 (1H, s), 7.78 (1H, d), 7.54 (2H, q), 7.36 (1H, d), 4.61 (2H, d), 3.64-3.48 (1H, m), 3.03 (2H, d), 3.04-2.94 (2H, m), 1.96 (3H, s), 1.97-1.91 (2H, m), 1.76-1.48 (14H, m).

MS: APCI(+ve) 569 (M+H⁺).

### EXAMPLE 23

### 2-[6-Chloro-5-[[(tricyclo[3.3.1.1^{3,7}]dec-1-ylmethyl)amino]carbonyl]-2-quinolinyl]-benzoic acid

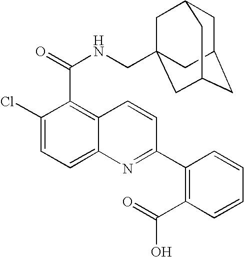

To 2,6-dichloro-N-(tricyclo[3.3.1.1^{3,7}]dec-1-ylmethyl)-5-quinolinecarboxamide (prepared as described in WO200308579) (200 mg) in tetrahydrofuran (1 mL) and water (1 mL) was added tetrakis(triphenylphosphine)palladium (10 mg), sodium carbonate (109 mg) and 2-(4,4,5,5-tetramethyl-1,3,2-dioxaborolan-2-yl)-benzoic acid ethyl ester (142 mg). The mixture was heated in a microwave for 120 minutes at 110° C. and then cooled to room temperature. Sodium hydroxide solution (48% w/v) (500 μL ) and methanol (2 mL) were added to the reaction which was then heated in a microwave at 70° C. for 1 h. The solvent was removed under vacuum and the reaction acidified with 2M hydrochloric acid. Removal of volatiles under vacuum was followed by dissolution in methanol and purification by HPLC (Symmetry—0.1% trifluoroacetic acid/acetonitrile) to afford the title compound (115 mg).

¹H NMR (400 MHz, d₆-DMSO) δ 8.72 (1H, t), 8.15 (1H, d), 8.01 (1H, d), 7.84 (3H, dd), 7.71-7.66 (2H, m), 7.63-7.56 (1H, m), 3.10 (2H, d), 1.97 (3H, s), 1.74-1.56 (12H, m).

MS: APCI(+ve) 475 (M+H⁺).

m.p. 156-158° C.

### EXAMPLE 24

### 3-[6-Chloro-5-[[(tricyclo[3.3.1.1^{3,7}]dec-1-ylmethyl)amino]carbonyl]-2-quinolinyl]-benzoic acid

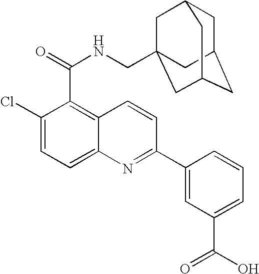

The title compound was prepared as described in Example 23 using 3-borono-benzoic acid methyl ester (92 mg). The compound was purified by HPLC (Symmetry—0.1% aqueous ammonium acetate/acetonitrile) to afford the title compound (50 mg).

¹H NMR (300 MHz, d₆-DMSO) δ 13.21 (1H, s), 8.86 (1H, s), 8.75-8.66 (1H, m), 8.52 (1H, d), 8.37 (1H, d), 8.24 (1H, d), 8.17 (1H, d), 8.10 (1H, d), 7.86 (1H, d), 7.71 (1H, t), 3.11 (2H, d), 1.98 (3H, s), 1.76-1.55 (12H, m).

MS: APCI(−ve) 473 (M−H⁺).

m.p. 297-298° C.

### EXAMPLE 25

### 4-[6-Chloro-5-[[(tricyclo[3.3.1.1^{3,7}]dec-1-ylmethyl)amino]carbonyl]-2-quinolinyl]-benzoic acid

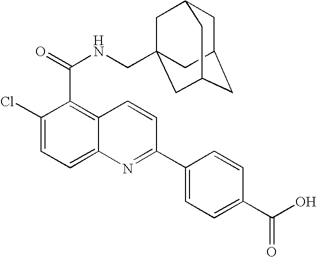

The title compound was prepared as described in Example 23 using 4-borono-benzoic acid methyl ester (92 mg). The compound was purified by HPLC (Symmetry—0.1% aqueous trifluoroacetic acid/acetonitrile) to afford the title compound (10 mg).

¹H NMR (400 MHz, d₆-DMSO) δ 13.14 (1H, s), 8.73-8.68 (1H, m), 8.42 (2H, d), 8.37 (1H, d), 8.24 (1H, d), 8.17-8.09 (2H, m), 7.87 (1H, d), 3.10 (2H, d), 1.98 (3H, s), 1.73-1.57 (12H, m).

MS: APCI(+ve) 475 (M−H⁺).

m.p. 316-317° C.

### EXAMPLE 26

### 1-[6-Chloro-5-[[(tricyclo[3.3.1.1^{3,7}]dec-1-ylmethyl)amino]carbonyl]-2-quinolinyl]-4-methyl-4-piperidinecarboxylic acid

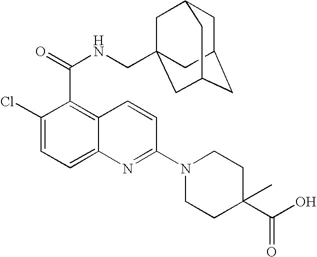

Prepared by the method of Example 3 using 2,6-dichloro-N-(tricyclo[3.3.1.1^{3,7}]dec-1-ylmethyl)-5-quinolinecarboxamide (prepared as described in WO2003080579) and ethyl 4-methyl-piperidine-4-carboxylate hydrochloride (the product of treatment of ethyl N-boc-4-methyl-4-piperidine carboxylate (prepared as described in WO9925685) with HCl/CH₂Cl₂ and concentrating in vacuo) followed by purification by preparative HPLC to afford the title compound (15 mg).

¹H NMR (400 MHz, d₆-DMSO) δ 8.26 (1H, s), 7.86 (1H, d), 7.73 (1H, d), 7.59 (1H, d), 7.40 (1H, d), 4.08 (2H, dt), 3.53-3.43 (2H), 3.06 (2H, d), 2.09 (2H, d), 1.97 (3H, s), 1.76-1.46 (14H, m), 1.22 (3H, s).

MS: APCI(+ve) 496 (M+H⁺).

### EXAMPLE 27

### N-[6-Chloro-5-[[(tricyclo[3.3.1.1^{3,7}]dec-1-ylmethyl)amino]carbonyl]-2-quinolinyl]-β-alanine

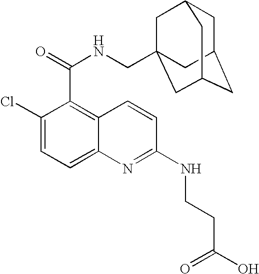

Prepared by the method of Example 2 using 2,6-dichloro-N-(tricyclo[3.3.1.1^{3,7}]dec-1-ylmethyl)-5-quinolinecarboxamide (prepared as described in WO2003080579) (250 mg), β-alanine 1,1-dimethylethyl ester hydrochloride (1 g) and triethylamine (0.9 ml), heating for 200 minutes at 140° C. Further purification by recrystallisation from methanol gave the title compound (66 mg).

¹H NMR (400 MHz, d₆-DMSO) δ 12.22 (1H, s), 8.50 (1H, t), 7.63 (1H, d), 7.49 (2H, dd), 7.30 (1H, t), 6.85 (1H, d), 3.59 (2H, dd), 3.02 (2H, d), 2.57 (2H, t), 1.95 (3H, s), 1.73-1.53 (12H, m).

MS: APCI(+ve) 442 (M+H⁺).

m.p. 251-252° C.

### EXAMPLE 28

### 5-[6-Chloro-5-[[(tricyclo[3.3.1.1^{3,7}]dec-1-ylmethyl)amino]carbonyl]-2-quinolinyl]-3-pyridinecarboxylic acid

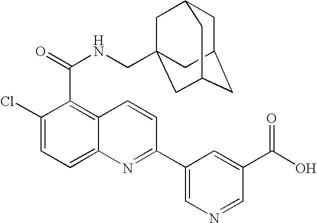

To 2,6-dichloro-N-(tricyclo[3.3.1.1^{3,7}]dec-1-ylmethyl)-5-quinolinecarboxamide (prepared as described in WO200308579) (200 mg) in tetrahydrofuran (2 mL) and water (1 mL) was added tetrakis(triphenylphosphine)palladium (40 mg), sodium carbonate (652 mg), 5-bromo-3-pyridinecarboxylic acid (333 mg) and 4,4,4′,4′,5,5,5′,5′-octamethyl-2,2′-bi-1,3,2-dioxaborolane (390 mg). The mixture was heated in a microwave for 150 minutes at 150° C. and then cooled to room temperature. Water was added to the reaction mixture which was subsequently extracted 4 times with dichloromethane. The combined organics were washed with brine, dried over magnesium sulfate, filtered and concentrated in vacuo. The crude product was dissolved in methanol and loaded onto a Varian NH₂ cartridge, washed with methanol and was eluted using 10% acetic acid in methanol. Further purification by preparative HPLC (Symmetry—0.1% aqueous trifluoroacetic acid/acetonitrile) gave the title compound (21 mg).

¹H NMR (400 MHz, d₆-DMSO) δ 9.67 (1H, d), 9.20 (1H, d), 9.10 (1H, t), 8.75 (1H, t), 8.49 (1H, d), 8.26 (1H, d), 8.20 (1H, d), 7.90 (1H, d), 3.11 (2H, d), 1.98 (3H, s), 1.73-1.58 (12H, m).

MS: APCI(−ve) 474 (M−H⁺).

m.p. 247° C.

Pharmacological Analysis

Certain compounds such as benzoylbenzoyl adenosine triphosphate (bbATP) are known to be agonists of the P2X₇ receptor, effecting the formation of pores in the plasma membrane (Drug Development Research (1996), 37(3), p. 126). Consequently, when the receptor is activated using bbATP in the presence of ethidium bromide (a fluorescent DNA probe), an increase in the fluorescence of intracellular DNA-bound ethidium bromide is observed. The increase in fluorescence can be used as a measure of P2X₇ receptor activation and therefore to quantify the effect of a compound on the P2X₇ receptor.

In this manner, each of the title compounds of the Examples was tested for antagonist activity at the P2X₇ receptor. Thus, the test was performed in 96-well flat bottomed microtitre plates, the wells being filled with 250 μl of test solution comprising 200 μl of a suspension of THP-1 cells (2.5×10⁶ cells/ml) containing 10⁻⁴M ethidium bromide, 25 μl of a high potassium buffer solution containing 10⁻⁵M bbATP, and 25 μl of the high potassium buffer solution containing concentrations of test compound typically from 30 μM -0.001 μM. The plate was covered with a plastics sheet and incubated at 37° C. for one hour. The plate was then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) were used separately in the test as controls. From the readings obtained, a pIC₅₀ figure was calculated for each test compound, this figure being the negative logarithm of the concentration of test compound necessary to reduce the bbATP agonist activity by 50%. Each of the compounds of the Examples demonstrated antagonist activity, having a pIC₅₀ figure >5.0. For example, the following table shows the pIC₅₀ figures for a representative selection of compounds:

| Compound of | |
|---|---|
| Example No. | pIC₅₀ |
| 3 | 7.1 |
| 12 | 7.8 |
| 13 | 7.6 |
| 24 | 7.2 |

1. A compound of formula 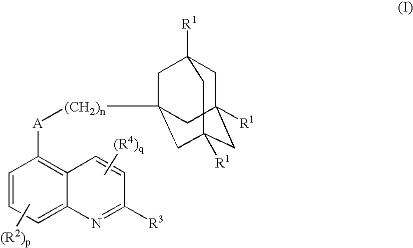
or a pharmaceutically acceptable salt or an in vivo hydrolysable ester thereof, wherein n represents 1, 2, or 3;
each R¹ independently represents hydrogen, hydroxy or a halogen;
A is C(O)NH or NHC(O);
p is 0, 1 or 2;
each R² independently represents halogen or C₁₋₆ alkyl optionally substituted by at least one substituent independently selected from hydroxyl, halogen, and C₁₋₆ alkoxy;
q is 0, 1 or 2;
each R⁴ independently represents halogen or C₁₋₆ alkyl optionally substituted by at least one substituent independently selected from hydroxyl, halogen and C₁₋₆alkoxy;
R³ represents a group Y¹R⁶ or Z¹R¹⁰;
R⁶ represents a group R⁸ or a 4- to 9-membered carbocyclic or heterocyclic ring, which carbocyclic or heterocyclic ring is substituted by at least one substituent independently selected from Y²R⁹ and Z²R¹¹, which 4- to 9-membered carbocyclic or heterocyclic ring may further be optionally substituted by at least one substituent independently selected from halogen, hydroxyl, C₁₋₆alkoxy, C₁₋₆ alkyl, phenyl and a 5- to 6-membered heteroaromatic ring, which C₁₋₆ alkyl, phenyl, or 5- to 6-membered heteroaromatic ring may be optionally substituted by at least one substituent selected from halogen, hydroxyl, and C₁₋₆alkoxy;
R⁸ and R⁹ each independently represent tetrazolyl, 1,2,3-triazolyl, 1,2,4-triazolyl or a 5- to 6-membered heterocyclic ring comprising from 1 to 4 heteroatoms independently selected from nitrogen, oxygen, and sulphur, which heterocyclic ring is substituted by at least one substituent selected from hydroxyl, ═O and ═S, and which heterocyclic ring may further be optionally substituted by at least one substituent selected from halogen, nitro, amino, cyano, C₁₋₆ alkylsulphonyl, C₁₋₆alkoxycarbonyl and a C₁₋₆ alkyl group which C₁₋₆ alkyl group can be optionally substituted by at least one substituent selected from halogen, hydroxyl, and amino;
R¹⁰ and R¹¹ each independently represent carboxyl, C₁₋₆ alkylsulphonylaminocarbonyl, C(O)NHOH or NHR¹²; R¹² represents CN, C₁₋₆ alkylsulphonyl, C₁₋₆ alkylcarbonyl, C₁₋₆ alkoxycarbonyl, C₁₋₆ alkylaminosulphonyl or (di)-C₁₋₆ alkylaminosulphonyl;
Y^{1 and Y}² each independently represent a bond, O, S(O)₀₋₂, NR⁷C(O), C(O)NR⁷, SO₂NR⁷, NR⁷SO₂, >NR⁷, O(CH₂)₁₋₆, S(O)₀₋₂(CH₂)₁₋₆, NR⁷(CH₂)₁₋₆, CH₂)₁₋₃O(CH₂)₁₋₃, CH₂)₁₋₃S(O)₀₋₂CH₂)₁₋₃, (CH₂)₁₋₃NR⁷(CH₂)₁₋₃, CH₂)₁₋₃NR⁷C(O)(CH₂)₀₋₃, (CH₂)₁₋₃C(O)NR⁷(CH₂)₀₋₃, S(O)₀₋₂(CH₂)₁₋₆NR⁷ or C₁₋₆ alkylene which C₁₋₆ alkylene can be optionally substituted by at least one substituent independently selected from hydroxyl, halogen, and C₁₋₆alkoxy;
Z¹ and Z² each independently represent a bond, O(CH₂)₁₋₆, S(O)₀₋₂(CH₂)₁₋₆, NR⁷(CH₂)₁₋₆, (CH₂)₁₋₃O(CH₂)₁₋₃, (CH₂)₁₋₃S(O)₀₋₂(CH₂)₁₋₃, (CH₂)₁₋₃NR⁷(CH₂)₁₋₃, (CH₂)₁₋₃NR⁷C(O)(CH₂)₁₋₃, (CH₂)₁₋₃, (CH₂)₁₋₃C(O)NR⁷(CH₂)₁₋₃ or C₁₋₆ alkylene which C₁₋₆ alkylene can be optionally substituted by at least one substituent independently selected from hydroxyl, halogen and C₁₋₆alkoxy; and
each R⁷ independently represents hydrogen or a C₁₋₆ alkyl group which can be optionally substituted by at least one substituent independently selected from hydroxyl, halogen and C₁₋₆alkoxy;
with the provisos that;
(a) when R³ represents Y¹R⁶ and Y¹ represents NR⁷(CH₂)₁₋₆, S(CH₂)₁₋₆, O(CH₂)₁₋₆ or an optionally substituted C₁₋₆ alkylene, then R⁶ does not represent oxopyrrolidinyl;
(b) when R¹ represents Z¹R¹⁰ and Z¹ represents (CH₂)₁₋₃NR⁷(CH₂)₁₋₃, then R¹⁰ does not represent carboxyl;
(c) when R³ represents Y¹R⁶ and Y¹ represents (CH₂)₁₋₃NR⁷(CH₂)₁₋₃ and R⁶ represents a group R⁸ then R⁸ does not represent a 5- to 6-membered heterocyclic ring substituted by hydroxyl or ═O;
(d) when R³ represents Y¹R⁶ and Y¹ represents (CH₂)₁₋₃NR⁷(CH₂)₁₋₃ and R⁶ represents a 4- to 9-membered carbocyclic or heterocyclic ring substituted by Z²R¹¹ and Z² represents a bond, then R¹¹ does not represent carboxyl;
(e) when R³ represents Y¹R⁶ and Y¹ represents NR⁷CH₂)₁₋₆, S(CH₂)₁₋₆, O(CH₂)₁₋₆ or an optionally substituted C₁₋₆ alkylene and R⁶ represents phenyl substituted by Z²R¹¹ and Z² represents a bond, then R¹¹ does not represent C₁₋₆ alkylsulphonylamino;
(f) when R³ represents Z¹R¹⁰ and Z¹ represents O(CH₂)₁₋₆, S(CH₂)₁₋₆, NR⁷(CH₂)₁₋₆ or an optionally substituted C₁₋₆ alkylene and R¹⁰ represents NHR¹² , then R¹² does not represent C₁₋₆ alkylcarbonyl; and
(g) the compound is not selected from tert-butyl 1-{5-[(1-adamantylacetyl)amino]-6-methylquinolin-2-yl}piperidin-4-ylcarbamate and tert-butyl (3S)-1-{5-[(1-adamantylacetyl)amino]-6-methylquinolin-2-yl}pyrrolidin-3-ylcarbamate.

2. A compound according to claim 1, wherein A represents C(O)NH.

3. A compound according to claim 1, wherein R¹⁰ and R¹¹ each independently represent carboxyl, C₁₋₆ alkylsulphonylaminocarbonyl or C(O)NHOH.

4. A compound according to claim 1, wherein R³ represents a group Y¹R⁶.

5. A compound according to claim 1, wherein Y¹ represents a bond.

6. A compound according to claim 1, wherein R⁶ represents an aliphatic 5- to 8-membered heterocyclic ring containing one nitrogen atom and optionally one further heteroatom selected from nitrogen and oxygen.

7. A compound according to claim 1, which is selected from
1-[6-Chloro-5-[(tricyclo[3.3.1.1^{3,7}]dec-1-ylacetyl)amino]-2-quinolinyl]-4piperidinecarboxylic acid,
1-[6-Chloro-5-[[(tricyclo[3.3.1.1^{3,7}]dec-1-ylmethyl)amino]carbonyl]-2-quinolinyl]-D-proline,
1-[6-Chloro-5-[[(tricyclo[3.3.1.1^{3,7}]dec-1-ylmethyl)amino]carbonyl]-2-quinolinyl]-(3R)-3-piperidinecarboxylic acid,
6-Chloro-2-[4-(1,5-dihydro-5-oxo-4H-1,2,4-triazol-4-yl)-1-piperidinyl]-N-(tricyclo[3.3.1.1^{3,7}]dec-1-ylmethyl)-5-quinolinecarboxamide,
4-[6-Chloro-5-[[(tricyclo[3.3.1.1^{3,7}]dec-1-ylmethyl)amino]carbonyl]-2-quinolinyl]-1-piperazineacetic acid,
6-Chloro-2-[(3S)-3-[[2-(2H-tetrazol-5yl)ethyl]amino]-1-pyrrolidinyl]-N-(tricyclo[3.3.1.1^{3,7}]dec-1-ylmethyl)-5-quinolinecarboxamide,
1-[6-Chloro-5-[[(tricyclo[3.3.1.1^{3,7}]dec-1-ylmethyl)amino]carbonyl]-2-quinolinyl]-4-piperidinecarboxylic acid,
1-[6-Chloro-5[[(tricyclo[3.3.1.1^{3,7}]dec-1-ylmethyl)amino]carbonyl]-2-quinolinyl]-4-piperidineacetic acid,
6-Chloro-2-[4-(2H-tetrazol-5yl)butyl]-N-(tricyclo[3.3.1.1^{3,7}]dec-1-ylmethyl)-5-quinolinecarboxamide,
6-Chloro-2-[4-(4,5-dihydro-5oxo-1,2,4-oxadiazol-3-yl)butyl]-N-(tricyclo[3.3.1.1^{3,7}]dec-1-methyl)-5-quinolinecarboxamide,
N-[(3S)-1-[6-Chloro-5-[[(tricyclo[3.3.1.1^{3,7}]dec-1-ylmethyl)amino]carbonyl]-2-quinolinyl]-3-pyrrolidinyl]-β-alanine,
N-[(3-S)-1-[6-Chloro-5-[[(tricyclo[3.3.1.1^{3,7}]dec-1-ylmethyl)amino]carbonyl]-2-quinolinyl]-3-piperidinyl]-β-alanine,
6-Chloro-2-[(3S)-3-[[2-(2H-tetrazol-5yl)ethyl]amino]-1-piperidinyl]-N-(tricyclo[3.3.1.1^{3,7}]dec-1-ylmethyl)-5-quinolinecarboxamide,
6-Chloro-2-[(3S)-3-[methyl[2-(2H-tetrazol-5-yl)ethyl]amino]-1-pyrrolidinyl]-N-(tricyclo[3.3.1.1^{3,7}]dec-1-ylmethyl)-5-quinolinecarboxamide,
4-[6-Chloro-5-[[(2-tricyclo[3.3.1.1^{3,7}]dec-1-ylethyl)amino]carbonyl]-2-quinolinyl]-1-piperazinepropanoic acid,
1-[6-Chloro-5-[[(tricyclo[3.3.1.1^{3,7}]dec-1-ylmethyl)amino]carbonyl]-2-quinolinyl]-4-hydroxy-4-piperidinecarboxylic acid,
1-[6-Chloro-5-[[(tricyclo[3.3.1.1^{3,7}]dec-1-ylmethyl)amino]carbonyl]-2-quinolinyl]-4-phenyl-4-piperidinecarboxylic acid,
(1R,5S)-3-[6-Chloro-5-[[(tricyclo[3.3.1.1^{3,7}]dec-1-ylmethyl)amino]carbonyl]-2-quinolinyl]-3-azabicyclo[3.1.0]hexane-6-carboxylic acid,
6-Chloro-2-[4-[[(methylsulfonyl)amino]carbonyl]-1-piperidinyl]-N-(tricyclo[3.3.1.1^{3,7}]dec-1-ylmethyl)-5-quinolinecarboxamide,
6-Chloro-2-[4-hydroxyamino)carbonyl]-1-piperidinyl]-N-(tricyclo[3.3.1.1^{3,7}]dec-1-ylmethyl)-5-quinolinecarboxamide,
6-Chloro-2-[4-(1H-1,2,4-triazol-3-ylsulfonyl)-1-piperidinyl]-N-(tricyclo[3.3.1.1^{3,7}]dec-1-ylmethyl)-5-quinolinecarboxamide,
2-[6-Chloro-5-[[(tricyclo[3.3.1.1^{3,7}]dec-1-ylmethyl)amino]carbonyl]-2-quinolinyl]-benzoic acid,
3-[6-Chloro-5-[[(tricyclo[3.3.1.1^{3,7}]dec-1-ylmethyl)amino]carbonyl]-2-quinolinyl]-benzoic acid,
4-[6-Chloro-5[[(tricyclo[3.3.1.1^{3,7}]dec-1-ylmethyl)amino]carbonyl]-2-quinolinyl]-benzoic acid,
1-[6-Chloro-5-[[(tricyclo[3.3.1.1^{3,7}]dec-1-ylmethyl)amino]carbonyl]-2-quinolinyl]-4-methyl-4-piperidinecarboxylic acid,
N-[6-Chloro-5-[[(tricyclo[3.3.1.1^{3,7}]dec-1-ylmethyl)amino]carbonyl]-2-quinolinyl]-β-alanine,
5-[6-Chloro-5-[[(tricyclo[3.3.1.1^{3,7}]dec-1-ylmethyl)amino]carbonyl]-2-quinolinyl]-3-pyridinecarboxylic acid,
or a pharmaceutically acceptable salt or an in vivo hydrolysable ester thereof.

8. A process for the preparation of a compound of formula (I) according to claim 1 or a pharmaceutically acceptable salt or an in vivo hydrolysable ester thereof, which comprises either:
(a) reacting a compound of formula (III) 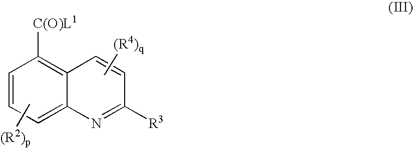
wherein L¹ represents a leaving group (e.g. hydroxyl or halogen) and R², R³, R⁴, p and q are as defined in formula (I), with a compound of formula (IV), 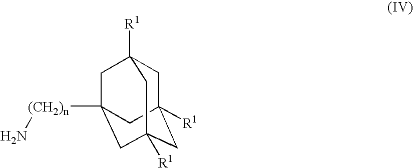
wherein R¹ and n are as defined in formula (I); or
(b) reacting a compound of formula (V) 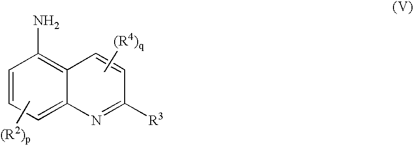
wherein R², R³, R⁴, p and q are as defined in formula (I), with a compound of formula (VI) 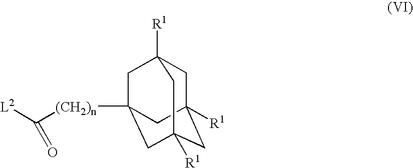
wherein L² represents a leaving group (e.g. hydroxoyl or halogen) and R¹ and n are as defined in formula (I); or
(c) when R³ represents a group Y¹R⁶ or Z¹R¹⁰ wherein the atom directly attached to the quinoline group of formula (I) is a nitrogen atom, reacting a compound of formula (VII) 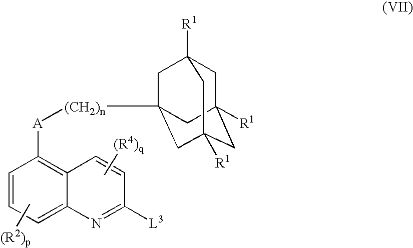
wherein L³ is a leaving group (e.g. halogen, paratoluene sulphonate or methane sulphonate), and all other variables are as defined in relation to formula (I), with a compound of formula (VIII), H—NY^{1″}R^{6″} or formula (IX), H—NZ^{1″}R^{10″} wherein NY^{1″}R^{6″} or NZ^{1″}R^{10″} make up a group of Y¹R⁶ or Z¹R¹⁰ respectively as defined in formula (I); or
(d) when R³ represents a group Y¹R⁶ or Z¹R¹⁰ wherein the group directly attached to the quinoline group of formula (I) is CH₂CH₂, reacting a compound of formula (VII) as defined in (c) above with a compound of formula (X), (XI), (XII) or (XIII) 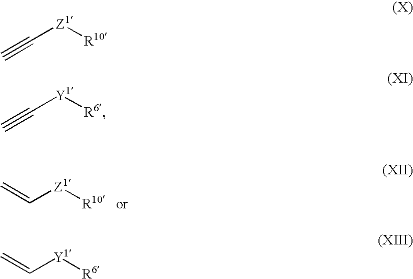
wherein Y^{1′}R^{6′} and Z^{1′}R^{10′} are suitably defined such that reaction of (VII) with (X), (XI), (XII) or (XIII) and subsequent hydrogenation of any resulting alkene or alkyne yields a compound wherein R³ represents a group Y¹R⁶ or Z¹R¹⁰; or
(e) when R³ represents a group Y¹R⁶ or Z¹R¹⁰ wherein the group directly attached to the quinoline group of formula (I) is CH₂CH₂N, reacting a compound of formula (VII) as defined in (c) above with a compound of formula (XIV) 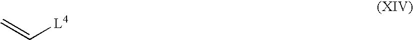
wherein L⁴ is a leaving group (eg. trialkyltin, dialkyboron or zinc), followed by reaction with a compound of formula (XV), HNY^{1′″}R^{6′″} or (XVI) HZ^{1′″}R^{10′″}, wherein NY^{1′″}R^{6′″} or NZ^{1′″}R^{10′″} make up a group of Y¹R⁶ or Z¹R¹⁰ respectively as defined in formula (I);
(f) when R³ represents a group Y¹R⁶ or Z¹R¹⁰ wherein the group directly attached to the quinoline group of formula (I) is CH₂N, reacting a compound of formula (VII) as defined in (c) above with a compound of formula (XIV) as defined in (e) above, followed by an oxidation reaction and then by reaction with a compound of formula (XV) or (XVI) as defined in (e) above under reductive amination conditions; or
(g) when R³ represents a group Y¹R⁶ or Z¹R¹⁰ wherein the group directly attached to the quinoline group of formula (I) is CH₂CH₂, reacting a compound of formula (VII) as defined in (c) above with a compound of formula (XII) or (XIII) as defined above wherein Y^{1′}R^{6′}, Z^{1′}R^{10′} are suitably defined such that saturation of alkene and subsequent combination with a compound of formula (VII) yields a compound wherein R³ represents a group Y¹R⁶ or Z¹R¹⁰; or
(h) when R⁸ or R⁹ represents a tetrazole, reacting a compound of formula 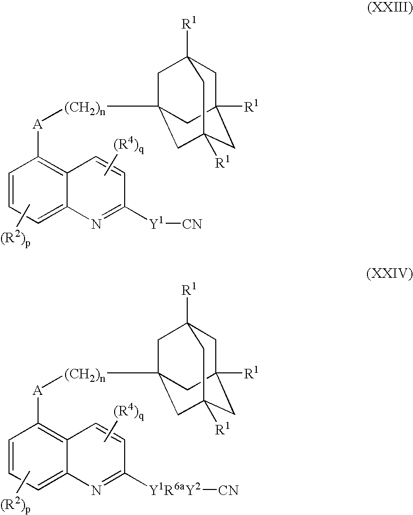
wherein R^{6a} is 4- to 9-membered carbocyclic or heterocyclic ring and all other variables are as defined in relation to formula (I), with a compound of formula PN₃ wherein P is sodium, a trialkylsilyl, an alkyltin or ammonium; or
(i) when R⁸ or R⁹ represents a group of formula (XVII) 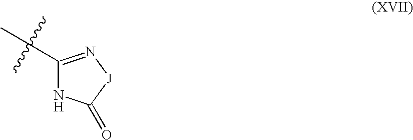
reacting a compound of formula (XXIII) or (XXIV) as defined above in (h) with hydroxylamine, followed by treatment with 1,1′-thiocarbonyldiimidazole and subsequent treatment with silica to yield a compound wherein R⁸ and R⁹ represent a group of formula (XVII) wherein J is S; alternatively reacting a compound of formula (XXVII) or (XXVIII) with hydroxylamine, followed by treatment with a chloroformate to yield a compound wherein R⁸ or R⁹ represent a group of formula (XVII) wherein J is O; or
(j) when R⁸ and R⁹ represent a group of formula (XVIII) 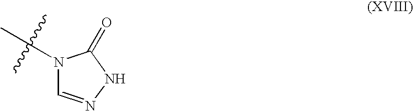
reacting a compound of formula 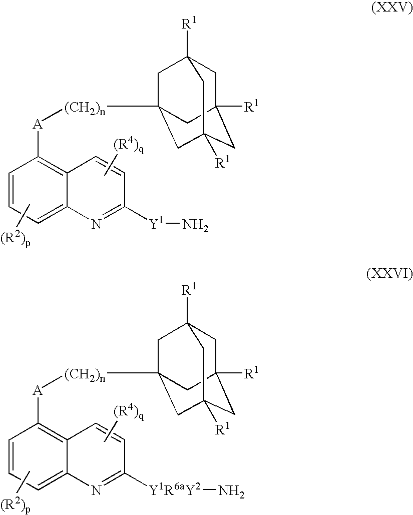
wherein R^{6a} is a 4- to 9-membered carbocyclic or heterocyclic ring and all other variables are as defined in relation to formula (I), with phosgene or a phosgene equivalent followed by treatment with formyl hydrazine and subsequent treatment with base; or
(k) when R⁸ and R⁹ represent a group of formula (XIX) 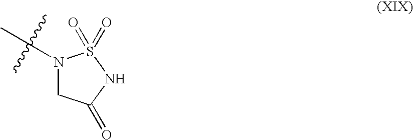
reacting a compound of formula (XXV) or (XXVI) as defined above in (j) with ethyl chloroacetate, followed by reaction with (chlorosulfonyl)-carbamic acid, 1,1-dimethylethyl ester and subsequent treatment with acid and base to yield a compound wherein R⁸ or R⁹ represent a group of formula (XIX); or
(l) when R₃ represents a group Y¹R⁶ wherein Y¹ is a bond and R⁶ is an aromatic carbocyclic or heterocyclic ring substituted by carboxyl, reacting a compound of formula (VII) as defined in (c) above with a compound of formula (XXVII) 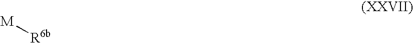
(XXVII)
wherein M represents an an organoboron group such as B(OH)₂, B(O^{j}PR)₂, BEt₂ or boronic acid pinacol cyclic ester and R^{6b} represents an aromatic carbocyclic or heterocyclic ring substituted by carboxyl, or CO₂C₁₋₆alkyl, optionally followed by reaction with a base such; or
(m) when R₃ represents a group Y¹R⁶ wherein Y¹ is a bond and R⁶ is an aromatic carbocyclic or heterocyclic ring substituted by carboxyl, reacting a compound of formula (VII) as defined in (c) above with a compound of formula (XXVII) 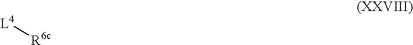
wherein L⁴ represents a leaving group and R^{6c} represents an aromatic carbocyclic or heterocyclic ring substituted by carboxyl, in the presence of a diboron compound;
and optionally after (a), (b), (c), (d), (e), (f), (g), (h), (i), (j), (k), (l), or (m) carrying out one or more of the following:
converting the compound obtained to a further compound of the invention
forming a pharmaceutically acceptable salt of the compound
forming an in vivo hydrolysable ester of the compound.

9. A pharmaceutical composition comprising a compound of formula (I), or a pharmaceutically acceptable salt or an in vivo hydrolysable ester thereof, as claimed in claim 1 in association with a pharmaceutically acceptable adjuvant, diluent or carrier.

10. A process for the preparation of a pharmaceutical composition comprising a compound of formula (I), or a pharmaceutically acceptable salt or an in vivo hydrolysable ester thereof as claimed in claim 1, in association with a pharmaceutically acceptable adjuvant, diluent or carrier, which comprises mixing a compound of formula (I), or a pharmaceutically acceptable salt or an in vivo hydrolysable ester thereof, with a pharmaceutically acceptable adjuvant, diluent or carrier.

11-13. (canceled)

14. A method of treating asthma or chronic obstructive pulmonary disease, the method comprising administering to a patient a therapeutically affective amount of a compound of formula (I), or a pharmaceutically acceptable salt or an in vivo hydrolysable ester thereof, as claimed in claim 1.

15. (canceled)

16. A method of treatment rheumatoid arthritis which comprises administering to a patient a therapeutically effective amount of a compound of formula (I), or a pharmaceutically acceptable salt or an in vivo hydrolysable ester thereof, as claimed in claim 1.

17. A method of treating osteoarthritis which comprises administering to a patient a therapeutically effective amount of a compound of formula (I), or a pharmaceutically acceptable salt or an in vivo hydrolysable ester thereof, as claimed in claim 1.

18. A method of treating an obstructive airways disease which comprises administering to a patient a therapeutically effective amount of a compound of formula (I), or a pharmaceutically acceptable salt or an in vivo hydrolysable ester thereof, as claimed in claim 1.

19. A method of treating atherosclerosis which comprises administering to a patient a therapeutically effective amount of a compound of formula (I), or a pharmaceutically acceptable salt or an in vivo hydrolysable ester thereof, as claimed in claim 1.
